(19) United States
(12) Patent Application Publication
(10) Pub. No.: US 2025/0164478 A1
(43) Pub. Date: May 22, 2025

(54) Methods and Compositions For Measuring Serum Analyte Levels From Biological Matrices

(71) Applicant: ABREOS BIOSCIENCES, INC., La Jolla, CA (US)

(72) Inventors: Bradley T. Messmer, La Jolla, CA (US); Lesley Page, La Jolla, CA (US)

(21) Appl. No.: 18/840,249

(22) PCT Filed: Mar. 2, 2023
(86) PCT No.: PCT/US2023/063591

§ 371 Date: Aug. 21, 2024

Related U.S. Application Data
(60) Provisional application No. 63/315,935, filed on Mar. 2, 2022.

(51) Int. Cl. G01N 33/543; A61B 5/15; A61B 10/00; G01N 33/569; G01N 33/577

(52) U.S. Cl. CPC G01N 33/54388; A61B 5/150045; A61B 10/0051; G01N 33/56983; G01N 33/577; G01N 2333/165; G01N 2469/20

(57) ABSTRACT

Provided herein are novel methods and compositions for determining serum analyte levels from biological matrices. The methods disclosed herein include determining a level of a protein in a serum of a patient in need thereof, comprising: (a) perturbing a soft tissue in a mouth of the patient: (b) collecting a sample of fluid from the oral, nasal, or other cavity of the patient; and (c) measuring the level of the protein in the sample of fluid from the cavity of the patient, wherein measuring the level of one or more protein in the sample of fluid from the sample of the patient affords a normalization or calibration curve allowing an accurate measurement of the level(s) of the desired analyte(s) in the serum of the patient.

### CROSS-REFERENCE

This application claims the benefit of U.S. Provisional Application No. 63/315,935 filed on Mar. 2, 2022, which is incorporated herein by reference in its entirety.

### BACKGROUND OF THE INVENTION

Accurate measurement of analytes in serum, including administered drugs, proteins, biomarkers, antibodies, anti-drug antibodies, etc. is critical for optimal patient treatment. Drug levels can vary enormously among people given the same standard dose. Insufficient dosing can result in a poor response to treatment, whereas excessive dosing results in higher costs, wasted resources, and troublesome side effects. Measurement of serum proteins, including administered drugs, is commonly performed by drawing a sample of a patient's blood. However, blood draws can be painful and inconvenient for a patient. Moreover, the importance of easily accessible, non-invasive samples such as saliva and nasal specimens, to effectively monitor serum antibody levels has been underscored by the Sars-CoV-2 (COVID 19) pandemic. As such, a need exists for improved methods and compositions for measuring serum proteins and analytes of interest and for accurately determining serum antibody levels from non-invasive samples (e.g., saliva and/or nasal samples).

### SUMMARY OF THE INVENTION

Disclosed herein are methods of determining a level of an analyte in a serum of a patient in need thereof, comprising: (a) perturbing a soft tissue in a mouth or other cavity of the patient; (b) collecting a sample of fluid from the mouth or other cavity of the patient; and (c) measuring the level of the analyte in the sample of fluid from the mouth or other cavity of the patient, wherein measuring the level of the analyte in the sample of fluid from the mouth or other cavity of the patient determines the level of the analyte in the serum of the patient. In some embodiments, the measuring the level of the analyte in the sample of fluid from the mouth or other cavity of the patient further comprises normalizing the level of the analyte to total IgG concentration in the sample of fluid from the mouth or other cavity of the patient. In some embodiments, the soft tissue in the mouth of the patient comprises the gums. In some embodiments, the other cavity of the patient comprises a nostril. In some embodiments, the soft tissue in the mouth of the patient is perturbed for 0-10 seconds, 10-30 seconds, 30 seconds-1 minute, or 1-2 minutes. In some embodiments, the soft tissue in the mouth of the patient is perturbed with an oral care tool or a part thereof. In some embodiments, the oral care tool comprises a manual toothbrush, an electric toothbrush, a retainer, an oral irrigator, a water flosser, a finger cot, a dental floss, a gum stimulator, a swab, a dental sponge, a swab stick, a form tip, an interdental brush, a dental scraper, a dental scaler, a dental pick, a dental stick, or a combination thereof. In some embodiments, wherein prior to perturbing the soft tissue in a mouth of the patient, the mouth of the patient is cleaned. In some embodiments, the mouth of the patient is cleaned with an oral care tool. In some embodiments, the analyte comprises an antibody or antigen-binding fragment thereof, biomarker, DNA, RNA, hormone, lipid, drug, anti-drug antibody or antigen-binding fragment thereof, other naturally occurring molecule, or a combination thereof. In some embodiments, the antibody or antigen-binding fragment thereof comprises a monoclonal antibody (mAb), a polyclonal antibody, a recombinant antibody, a chimeric antibody, a humanized antibody, a camelid antibody, a CDR-grafted antibody, F(ab)2, Fv, scFv, IgGΔCH2, F(ab′)2, scFv2CH3, F(ab), VL, VH, scFv4, scFv3, scFv2, dsFv, Fv, scFv-Fc, (scFv)2, a disulfide linked Fv, a single domain antibody (dAb), a diabody, a bispecific antibody, a multispecific antibody, a dual specific antibody, an anti-idiotypic antibody, a nanobody, a VHH antibody, IgA, IgD, IgE, IgG, IgM, a modified antibody, non-depleting IgG antibodies, T-bodies, Fc or Fab variants thereof. In some embodiments, the antibody or antigen-binding fragment thereof specifically binds to a SARS-CoV-2 spike protein. In some embodiments, the antibody or antigen-binding fragment thereof specifically binds to an Fc receptor. In some embodiments, the Fc receptor is an IgG Fc receptor. In some embodiments, the IgG Fc receptor is a neonatal Fc receptor. In some embodiments, the antibody or antigen-binding fragment thereof comprises adalimumab, infliximab, tocilizumab, vedolizumab, eculizumab, alemtuzumab, natalizumab, atezolizumab, bevacizumab, cetuximab, daratumumab, ipilimumab, nivolumab, obinutuzumab, pembrolizumab, pertuzumab, ramucirumab, rituximab, trastuzumab, golimumab, ustekinumab, denosumab, certolizumab pegol, secukinumab, alemtuzumab, blinatumomab, or antigen-binding fragment thereof. In some embodiments, the antibody or antigen-binding fragment thereof comprises natalizumab. In some embodiments, the anti-drug antibody or antigen-binding fragment thereof binds to a therapeutic antibody. In some embodiments, the anti-drug antibody or antigen-binding fragment thereof comprises an anti-idiotypic antibody to the therapeutic antibody. In some embodiments, the anti-idiotypic antibody comprises an antigen blocking anti-idiotype antibody, a non-blocking anti-idiotype antibody, or a complex specific anti-idiotype antibody.

Also provided herein are devices for determining a level of an analyte in the serum of a patient in need thereof, comprising: (a) an oral care tool or a part thereof; and a lateral flow assay test. In some embodiments, the oral care tool comprises a manual toothbrush, an electric toothbrush, a retainer, an oral irrigator, a water flosser, a finger cot, a dental floss, a gum stimulator, a swab, a dental sponge, a swab stick, a form tip, an interdental brush, a dental scraper, a dental scaler, a dental pick, a dental stick, or a combination thereof. In some embodiments, the analyte comprises an antibody or antigen-binding fragment thereof, biomarker, DNA, RNA, hormone, lipid, drug, anti-drug antibody or antigen-binding fragment thereof, other naturally occurring molecule, or a combination thereof. In some embodiments, the antibody or antigen-binding fragment thereof comprises a monoclonal antibody (mAb), a polyclonal antibody, a recombinant antibody, a chimeric antibody, a humanized antibody, a camelid antibody, a CDR-grafted antibody, F(ab)2, Fv, scFv, IgGΔCH2, F(ab′)2, scFv2CH3, F(ab), VL, VH, scFv4, scFv3, scFv2, dsFv, Fv, scFv-Fc, (scFv)2, a disulfide linked Fv, a single domain antibody (dAb), a diabody, a bispecific antibody, a multispecific antibody, a dual specific antibody, an anti-idiotypic antibody, a nanobody, a VHH antibody, IgA, IgD, IgE, IgG, IgM, a modified antibody, non-depleting IgG antibodies, T-bodies, Fc or Fab variants thereof. In some embodiments, the antibody or antigen-binding fragment thereof specifically binds to a SARS-CoV-2 spike protein. In some embodiments, the antibody or antigen-binding fragment thereof specifically binds to an Fc receptor. In some embodiments, the Fc receptor is an IgG Fc receptor. In some embodiments, the IgG Fc receptor is a neonatal Fc receptor.

The device of any one of claims 23-28, wherein the antibody or antigen-binding fragment thereof comprises adalimumab, infliximab, tocilizumab, vedolizumab, eculizumab, alemtuzumab, natalizumab, atezolizumab, bevacizumab, cetuximab, daratumumab, ipilimumab, nivolumab, obinutuzumab, pembrolizumab, pertuzumab, ramucirumab, rituximab, trastuzumab, golimumab, ustekinumab, denosumab, certolizumab pegol, secukinumab, alemtuzumab, blinatumomab, or antigen-binding fragment thereof. In some embodiments, the antibody or antigen-binding fragment thereof comprises natalizumab. In some embodiments, the anti-drug antibody or antigen-binding fragment thereof binds to a therapeutic antibody. In some embodiments, the anti-drug antibody or antigen-binding fragment thereof comprises an anti-idiotypic antibody to the therapeutic antibody. In some embodiments, the anti-idiotypic antibody comprises an antigen blocking anti-idiotype antibody, a non-blocking anti-idiotype antibody, or a complex specific anti-idiotype antibody.

Also provided herein are methods for treating a disease or condition in a subject in need thereof, comprising: (a) determining a level of an analyte in a serum of the patient, comprising: (i) perturbing a soft tissue in a mouth or other cavity of the patient; (ii) collecting a sample of fluid from the mouth or other cavity of the patient; and (iii) measuring the level of the analyte in the sample of fluid from the mouth or other cavity of the patient, wherein measuring the level of the analyte in the sample of fluid from the mouth or other cavity of the patient determines the level of the analyte in the serum of the patient; and (b) administering a drug to the subject. In some embodiments, the method further comprises adjusting a dosage of the drug after an initial administration of the drug. In some embodiments, the adjusting a dosage of the drug comprises increasing, decreasing, or maintaining the dosage of the drug. In some embodiments, the disease or condition comprises virus infection, bacterial infection, autoimmune disease, corona virus, multiple sclerosis, polyomavirus, JC virus, progressive multifocal leukoencephalopathy, Guillain-Barre syndrome, Myasthenia Gravis, flu, monkey pox, AIDS or a combination thereof. In some embodiments, the analyte comprises an antibody or antigen-binding fragment thereof, biomarker, DNA, RNA, hormone, lipid, drug, anti-drug antibody or antigen-binding fragment thereof, other naturally occurring molecule, or a combination thereof. In some embodiments, the antibody or antigen-binding fragment thereof comprises a monoclonal antibody (mAb), a polyclonal antibody, a recombinant antibody, a chimeric antibody, a humanized antibody, a camelid antibody, a CDR-grafted antibody, F(ab)2, Fv, scFv, IgGΔCH2, F(ab′)2, scFv2CH3, F(ab), VL, VH, scFv4, scFv3, scFv2, dsFv, Fv, scFv-Fc, (scFv)2, a disulfide linked Fv, a single domain antibody (dAb), a diabody, a bispecific antibody, a multispecific antibody, a dual specific antibody, an anti-idiotypic antibody, a nanobody, a VHH antibody, IgA, IgD, IgE, IgG, IgM, a modified antibody, non-depleting IgG antibodies, T-bodies, Fc or Fab variants thereof. In some embodiments, the antibody or antigen-binding fragment thereof specifically binds to a SARS-CoV-2 spike protein. In some embodiments, the antibody or antigen-binding fragment thereof specifically binds to an Fc receptor. In some embodiments, the Fc receptor is an IgG Fc receptor. In some embodiments, the IgG Fc receptor is a neonatal Fc receptor. In some embodiments, the antibody or antigen-binding fragment thereof comprises adalimumab, infliximab, tocilizumab, vedolizumab, eculizumab, alemtuzumab, natalizumab, atezolizumab, bevacizumab, cetuximab, daratumumab, ipilimumab, nivolumab, obinutuzumab, pembrolizumab, pertuzumab, ramucirumab, rituximab, trastuzumab, golimumab, ustekinumab, denosumab, certolizumab pegol, secukinumab, alemtuzumab, blinatumomab, or antigen-binding fragment thereof. In some embodiments, the antibody or antigen-binding fragment thereof comprises natalizumab. In some embodiments, in addition to or in lieu of measuring levels of an antibody drug, the patient's levels of anti-drug antibodies are measured.

Also provided herein are methods for treating a disease or condition in a subject in need thereof, comprising: (a) determining a level of an analyte in a serum of the patient, comprising: (i) perturbing a soft tissue in a mouth or other cavity of the patient; (ii) collecting a sample of fluid from the mouth or other cavity of the patient; and (iii) measuring the level of the analyte in the sample of fluid from the mouth or other cavity of the patient, wherein measuring the level of the analyte in the sample of fluid from the mouth or other cavity of the patient determines the level of the analyte in the serum of the patient; and (b) administering an anti-drug antibody or antigen-binding fragment thereof to the subject. In some embodiments, the anti-drug antibody or antigen-binding fragment thereof binds to a therapeutic antibody. In some embodiments, the anti-drug antibody or antigen-binding fragment thereof comprises an anti-idiotypic antibody to the therapeutic antibody. In some embodiments, the anti-idiotypic antibody comprises an antigen blocking anti-idiotype antibody, a non-blocking anti-idiotype antibody, or a complex specific anti-idiotype antibody.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

This patent application contains at least one drawing executed in color. Copies of this patent or patent application with color drawing(s) will be provided by the Office upon request and payment of the necessary fee.

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-1B are graphs showing that normalization of the saliva Spike S1 titer to total IgG present in saliva sample correlated to serum Spike S1 titer.

FIGS. 2A-2C provide graphs showing the obtained standard in WHO International Units (IU) using serum donations.

FIGS. 3A-3B are graphs showing similar levels of covid antibodies in saliva and serum when normalized to IgG levels.

FIGS. 4A-4B are graphs showing the correlation between actual serum Spike S1 IU and that predicted from saliva (FIG. 4A) and nasal fluid (FIG. 4B). The serum covid-19 antibody level was predicted from saliva (FIG. 4A, x axis) and nasal fluid (FIG. 4B, x axis) and plotted against the actual measured serum level (y axis). Both matrix types show good predictive ability for serum antibody levels. Trendline is forced through 0,0.

FIGS. 5A-5B are graphs illustrating serum (FIG. 5A) and saliva (FIG. 5B) predicted serum levels. Donor antibody levels (y axis) were tracked over time (x axis, days after vaccination). Red data points indicate patients who received a vaccination booster shot.

FIGS. 6A-6B are graphs illustrating serum (FIG. 6A) and nasal fluid (FIG. 6B) predicted serum levels. Donor antibody levels (y axis) were tracked over time (x axis, days after vaccination). Red data points indicate patients who received a vaccination booster shot.

FIG. 7 depicts that the mechanism of saliva collection can influence the amount of IgG obtained.

FIGS. 8A-8D depict that reducing the volume of buffer can cause an increase in antibody levels detected.

FIG. 9 depicts a comparison between serum vs saliva for a PRIMACOVID Covid-19 neutralizing antibody rapid test.

FIG. 10 depicts nasal fluid samples on a PRIMACOVID Covid-19 neutralizing antibody rapid test.

FIG. 11 illustrates that an infused therapeutic drug can be detected in saliva and nasal fluid.

FIGS. 12A-12C demonstrate an exemplary ELISA method.

FIG. 13 depicts an exemplary lateral flow device, including S1 protein on nitrocellulose (NC) and detection with anti-human IgG coupled gold nanoparticles.

FIGS. 14A-14B depicts the initial assessment of an exemplary lateral flow device format.

FIG. 15 depicts an exemplary collection device.

FIGS. 16A-16D depict that quantifiable levels of SARS-CoV-2 Spike S1 antibodies can be detected from both nasal and saliva samples.

FIGS. 17A-17D depict that normalization of SARS-CoV-2 antibody level to total IgG concentration in nasal and saliva samples can strengthen the correlation to serum antibody levels.

FIGS. 18A-18D depict the correlation between actual serum SARS-CoV-2 antibody and that predicted from saliva (FIGS. 18A and 18C) and nasal fluid (FIGS. 18B and 18D).

FIGS. 19A-19C depict that serum SARS-CoV-2 Spike S1 antibody levels and serum levels predicted from saliva and nasal samples decline over time.

FIGS. 20A-20H demonstrates the effect of IgG4 concentration on Bivalent NAT levels through the using veritope NTZ07 and the IgG calibration technique.

FIGS. 21A-21H demonstrates the use of saliva as a sample matrix and IgG calibration in monitoring levels of the MS (multiple sclerosis) antibody drug natalizumab and antibodies against COVID-19 to accurately predict serum levels for both analytes.

### DETAILED DESCRIPTION OF THE INVENTION

Traditionally, sample matrices such as blood, serum, plasma, or nasopharyngeal that diagnostic testing have relied on are often impractical, difficult, and/or even painful. Thus, there has been an increased interest in determining the utility and feasibility of non-invasive collections such as saliva and nasal specimens in diagnostics and correlating their biomarker levels with that found in serum.

The present disclosure provides methods and devices for measuring a level of an analyte in non-invasive samples (e.g., saliva and nasal specimens) of a patient to determine the serum analyte level of the patient. The present disclosure provides that measurements of analyte levels in non-invasive samples (e.g., saliva and nasal specimens) can be used to accurately predict serum levels by utilizing endogenous total IgG as an internal calibration. This method can be extended for the measurement of many different antibody analytes, making it of high interest for antibody therapeutic drug monitoring (TDM) applications.

In one aspect, provided herein, are methods of determining a level of a serum analyte in a patient in need thereof, comprising: (a) perturbing a soft tissue in a mouth or other cavity of the patient; (b) collecting a sample of fluid from the mouth or other cavity of the patient; and (c) measuring the level of the analyte in the sample of fluid from the mouth or other cavity of the patient, wherein measuring the level of the analyte in the sample of fluid from the mouth of the patient determines the level of the analyte in the serum of the patient. In some embodiments, the soft tissue in the mouth of the patient comprises the gums. In some embodiments, the other cavity of the patient comprises a nostril.

In another aspect, provided herein are devices for determining a level of an analyte in a serum of a patient. The devices comprise (a) an oral care tool or a part thereof and (b) a lateral flow assay test. The oral care tool or a part thereof may be used to perturb a soft tissue in the mouth of the patient and collect a sample of fluid from the mouth of the patient. The collected sample may flow into the lateral flow assay test in which the level of the analyte is measured. The methods and devices described herein can accurately predict the level of an analyte in a serum of a patient from non-invasive samples, such as saliva and nasal specimens. The methods and devices described herein can be used for monitoring many different analytes (e.g., antibodies, biomarkers, DNAs, RNAs, hormones, lipids, anti-drug antibodies, drugs, or other naturally occurring molecule) in a patient.

In another aspect, provided herein are methods for treating a disease or condition in a subject in need thereof, comprising (a) determining a level of an analyte in a serum of the patient, comprising: (i) perturbing a soft tissue in a mouth or other cavity of the patient: (ii) collecting a sample of fluid from the mouth or other cavity of the patient; and (iii) measuring the level of the analyte in the sample of fluid from the mouth or other cavity of the patient, wherein measuring the level of the analyte in the sample of fluid from the mouth or other cavity of the patient determines the level of the analyte in the serum of the patient; and (b) administering a drug to the subject. In some embodiments, in addition to or in lieu of measuring levels of an antibody drug, the patient's levels of anti-drug antibodies are measured.

In another aspect, provided herein are methods for treating a disease or condition in a subject in need thereof, comprising (a) determining a level of an analyte in a serum of the patient, comprising: (i) perturbing a soft tissue in a mouth or other cavity of the patient; (ii) collecting a sample of fluid from the mouth or other cavity of the patient; and (iii) measuring the level of the analyte in the sample of fluid from the mouth or other cavity of the patient, wherein measuring the level of the analyte in the sample of fluid from the mouth or other cavity of the patient determines the level of the analyte in the serum of the patient; and (b) administering an anti-drug antibody to the subject. In some embodiments, In some embodiments, the anti-drug antibody or antigen-binding fragment thereof comprises an anti-idiotypic antibody to the therapeutic antibody. In some embodiments, the anti-idiotypic antibody comprises an antigen blocking anti-idiotype antibody, a non-blocking anti-idiotype antibody, or a complex specific anti-idiotype antibody.

### Methods of Measuring a Level of a Serum Analyte

Disclosed herein, are methods of determining a level of a serum analyte (e.g., protein,) in a patient in need thereof, comprising: (a) perturbing a soft tissue in a mouth or other cavity of the patient; (b) collecting a sample of fluid from the mouth or other cavity of the patient; and (c) measuring the level of the protein in the sample of fluid from the mouth or other cavity of the patient, wherein measuring the level of the protein in the sample of fluid from the mouth or other cavity of the patient determines the level of the analyte in the serum of the patient. In some embodiments, the soft tissue in the mouth of the patient comprises gums. In some embodiments, the other cavity of the patient comprises a nostril. The method provides herein further comprise normalizing the level of the analyte to total IgG concentration in the sample of fluid from the mouth or other cavity of the patient. The normalization of the level of the analyte to total IgG concentration in the sample may lead to a high degree of correlation between the serum analyte level and the levels of the analyte in the mouth or other cavity of the patient.

Although the level of an analyte in saliva and/or nasal matrices may be lower than the serum analyte level, quantifiable levels of the analyte can be detected in the saliva and/or nasal matrices and show good correlation to serum analyte level. In some cases, the analyte (e.g., antibody) concentration measured in saliva and nasal samples may not track with serum due to physiological factors that affect the analyte concentration in a matrix dependent manner. For example, differences in an individual's hydration state or oral health may result in changes in saliva volume/viscosity and extent of analyte leakage, respectively. Total IgG concentration may be affected to the same extent as an antigen specific IgG, so total IgG is measured in the sample and used to correct for the effects of these matrix specific factors. Details of the correction calculation are explained in Table 1. Briefly, the ratio of specific IgG to total IgG is determined. The normalization of the level of the analyte to the total IgG concentration present in the sample can improve correlation between the level of analytes in saliva and/or nasal sample and the serum analyte level.

Both saliva and nasal specimens may provide good concordance with the predicted and measured serum antibody levels. Although saliva is considered a slightly more agreeable method of specimen collection, the concentration of the analyte detected in the sample may be very low. Adjustment in procedures, at the levels of sample collection and assay design may improve the concentration of the analyte and assay sensitivity.

In some embodiments, the methods described herein further comprise an IgG calibration step. The addition of an IgG calibration step may lead a much-improved correlation between the level of the analyte in saliva and/or nasal samples and the serum analyte level. Since the presence of IgG antibodies in saliva is due to passive leakage from blood (Subbarao K C, et al., J Pharm Bioallied Sci. 11 ((Suppl 2)), S135-139 (2019); Brandtzaeg O. J. Oral Microbiol. 5(2013), 1-24(2013)), the saliva/serum correlation can be extended to many types of serum analytes and allow for a much improved and simpler process to monitor levels.

The IgG calibration method described herein may be applied to different type of assay platforms. Examples of assay platforms include, but not limited to, enzyme-linked immunoassay (ELISA) and lateral flow assays (LFA). For example, quantitative lateral flow assays, using saliva as a matrix, would facilitate an easy, non-invasive method of sample collection along with rapidly generated results.

The methods described herein, in some embodiments, comprises perturbing the soft tissue in the mouth or other cavity of the patient for a sufficient time of period. This can help stimulate the tissue in and around the collection area, thereby producing more sample or a sample of better quality for collection. In some embodiments, perturbing the soft tissue in the mouth or other cavity of the patient comprises stimulating small amounts of blood release from the soft tissue in the mouth or other cavity of the patient. In some embodiments, the soft tissue in the mouth or other cavity of the patient are perturbed for at least 5, 10, 15, 20, 25, 30, 35, 40, 45, 50, 55, 60, 70, 80, 90, 100, 110, 120, 130, 140, or 150 seconds. In some embodiments, the soft tissue in the mouth or other cavity of the patient are perturbed for at most 5, 10, 15, 20, 25, 30, 35, 40, 45, 50, 55, 60, 70, 80, 90, 100, 110, 120, 130, 140, or 150 seconds. In some embodiments, the soft tissue in the mouth or other cavity of the patient are perturbed for at least 1, 2, 3, 4, 5, 6, 7, 8, 9, 10, 11, 12, 13, 14, 15, 16, 17, 18, 19, or 20 minutes. In some embodiments, the soft tissue in the mouth or other cavity of the patient are perturbed for at most 1, 2, 3, 4, 5, 6, 7, 8, 9, 10, 11, 12, 13, 14, 15, 16, 17, 18, 19, or 20 minutes. In some embodiments, the soft tissue in the mouth or other cavity of the patients are perturbed for at least 1 minute. In some embodiments, the soft tissue in the mouth or other cavity of the patients are perturbed for at least 2 minutes. In some embodiments, the soft tissue in the mouth or other cavity of the patients are perturbed for at most 1 minute. In some embodiments, the soft tissue in the mouth or other cavity of the patients are perturbed for at most 2 minutes. In some embodiments, the soft tissue in the mouth or other cavity of the patient are perturbed for 1-10 seconds, 10-30 seconds, 30 seconds-1 minute, or 1-2 minutes. In some embodiments, the soft tissue in the mouth or other cavity of the patient are perturbed for 1-10 seconds, 10-20 seconds, 10-20 seconds, 20-30 seconds, 30-40 seconds, 40-50 seconds, 50-60 seconds, 60-70 seconds, 70-80 seconds, 80-90 seconds, 90-100 seconds, 100-110 seconds, 110-120 seconds, 120-130 seconds, 130-140 seconds, 140-150 seconds. In some embodiments, the soft tissue in the mouth or other cavity of the patient are perturbed for 1-10 seconds, 10-30 seconds, 30-50 seconds, 50-70 seconds, 70-90 seconds, 90-110 seconds, 110-130 seconds, 130-150 seconds. In some embodiments, the soft tissue in the mouth or other cavity of the patient are perturbed for 1-30 seconds, 30-50 seconds, 50-70 seconds, 70-90 seconds, 90-110 seconds, 110-130 seconds, or 130-150 seconds. In some embodiments, the soft tissue in the mouth or other cavity of the patient are perturbed for 1-15 seconds, 15-30 seconds, 30-45 seconds, 45-60 seconds, 60-75 seconds, 75-90 seconds, 90-105 seconds, 105-120 seconds, 120-135 seconds, 130-150 seconds.

In some embodiments, the soft tissue in the mouth or other cavity of the patient is perturbed with a stimulating device. Examples of stimulating devices include, but not limited to, a manual toothbrush, electric toothbrush, retainer, oral irrigator, water flosser, dental floss, swab, bristles, or other devices that can stimulate the soft tissue in the mouth or other cavity of the patient. Prior to perturbing the soft tissue in the mouth or other cavity of the patient, the mouth or other cavity of the patient may be cleaned to remove undesired components that may interfere with the analysis. In some embodiments, a toothbrush and/or dental floss is used. In some embodiments, a swab and/or bristles is used. Other cleaning devices may also be used.

In some embodiments, the soft tissue in the mouth or other cavity of the patient is perturbed with an oral care tool or a part thereof. The oral care tool may include any commercial oral hygiene tools. Examples of the oral care tools include, but are not limited to, a manual toothbrush, an electric toothbrush, a retainer, an oral irrigator, a water flosser, a finger cot, a dental floss, a gum stimulator, a swab, a dental sponge, a swab stick, a form tip, an interdental brush, a dental scraper, a dental scaler, a dental pick, a dental stick, or a combination thereof.

In certain embodiments, prior to perturbing the soft tissue in a mouth of the patient, the mouth of the patient is cleaned. In some embodiments, the mouth of the patient is cleaned with an oral care tool. The oral care tool may include any commercial oral hygiene tools. Examples of the oral care tools include, but are not limited to, a manual toothbrush, an electric toothbrush, a retainer, an oral irrigator, a water flosser, a finger cot, a dental floss, a gum stimulator, a swab, a dental sponge, a swab stick, a form tip, an interdental brush, a dental scraper, a dental scaler, a dental pick, a dental stick, or a combination thereof. In some embodiments, the mouth of the patient is cleaned with mouthwash, mouth rinse, water, or any liquid.

The methods described herein comprises determining a level of an analyte in a serum of a patient. Examples of analytes include, but not limited to, an antibody or antigen-binding fragment thereof, biomarker, DNA, RNA, hormone, lipid, drug, anti-drug antibody or antigen-binding fragment thereof, other naturally occurring molecule or a combination thereof. In some embodiments, the analyte is an antibody. In some embodiments, the antibody is a monoclonal antibody, monovalent antibody, an intact antibody, a bivalent antibody, a scrambled antibody, a total antibody, a polyclonal antibody, a recombinant antibody, a chimeric antibody, a humanized antibody, a camelid antibody, a CDR-grafted antibody, F(ab)2, Fv, scFv, IgGΔCH2, F(ab′)2, scFv2CH3, F(ab), VL, VH, scFv4, scFv3, scFv2, dsFv, Fv, scFv-Fc, (scFv)2, a disulfide linked Fv, a single domain antibody (dAb), a diabody, a bispecific antibody, a multispecific antibody, a dual specific antibody, an anti-idiotypic antibody, a nanobody, a VHH antibody, IgA, IgD, IgE, IgG, IgM, a modified antibody, non-depleting IgG antibodies, T-bodies, Fc or Fab variants thereof. In some embodiments, the antibody is a mouse antibody or a human antibody.

In some embodiments, the antibody or antigen-binding fragment thereof described herein specifically binds to a SARS-CoV-2 spike protein. In some embodiments, the antibody or antigen-binding fragment thereof binds to S1 domain of the SARS-CoV-2 spike protein. In some embodiments, the antibody or antigen-binding fragment thereof binds to S2 domain of the SARS-CoV-2 spike protein.

In some embodiments, the antibody or antigen-binding fragment thereof described herein specifically binds to an Fc receptor. In some embodiments, the Fc receptor is an IgG Fc receptor. In some embodiments, the Fc receptor is an IgA Fc receptor. In some embodiments, the Fc receptor is an IgE Fc receptor. In some embodiments, the Fc receptor is an IgM Fc receptor.

In some embodiments, the IgG Fc receptor is a neonatal Fc receptor (FcRn). The FcRN plays a key role in regulating the life cycle of IgG and serum albumin. In some cases, FcRn extends the serum half-life of therapeutic antibodies. In some cases, FcRn extends the half-life of pathogenic IgG antibodies and promote autoimmune diseases. The antibodies or Fc fragments that have high binding affinity with FcRn can be used to treat autoimmune diseases (e.g. myasthenia gravis) by disrupting the binding of FcRn to pathogenic IgG antibodies.

In some embodiments, the antibody or antigen-binding fragment described herein comprises adalimumab, infliximab, tocilizumab, vedolizumab, eculizumab, alemtuzumab, natalizumab, atezolizumab, bevacizumab, cetuximab, daratumumab, ipilimumab, nivolumab, obinutuzumab, pembrolizumab, pertuzumab, ramucirumab, rituximab, trastuzumab, golimumab, ustekinumab, denosumab, certolizumab pegol, secukinumab, alemtuzumab, or blinatumomab. In some embodiments, the antibody or antigen-binding fragment described herein comprises alemtuzumab. In some embodiments, the antibody or antigen-binding fragment described herein comprises natalizumab.

In some embodiments, the antibody or antigen binding fragment described herein comprises a monoclonal antibody. Examples of monoclonal antibodies include, but are not limited to 3F8, Abagovomab, Abatacept, Abciximab, ACZ885, Adalimumab, Adecatumumab, Afelimomab, Aflibercept, Afutuzumab, Alacizumab, Alemtuzumab, Altumomab, Anatumomab, Anrukinzumab, Apolizumab, Arcitumomab, Aselizumab, Atlizumab, Atorolimumab, Bapineuzumab, Basiliximab, Bavituximab, Bectumomab, Belatacept, Belimumab, Bertilimumab, Besilesomab, Bevacizumab, Biciromab, Bivatuzumab, Blinatumomab, Canakinumab, Cantuzumab, Capromab, Catumaxomab, Cedelizumab, Certolizumab, Cetuximab Erbitux, Citatuzumab, Cixutumumab, Clenoliximab, CNTO 1275 (=ustekinumab), CNTO 148 (=golimumab), Conatumumab, Dacetuzumab, Dacliximab (=daclizumab), Daclizumab, Denosumab, Detumomab, Dorlimomab, Dorlixizumab, Ecromeximab, Eculizumab, Edobacomab, Edrecolomab, Efalizumab, Efungumab, Elsilimomab, Enlimomab, Epitumomab, Epratuzumab, Erlizumab, Ertumaxomab, Etanercept, Etaracizumab, Exbivirumab, Fanolesomab, Faralimomab, Felvizumab, Figitumumab, Fontolizumab, Foravirumab, Galiximab, Gantenerumab, Gavilimomab, Gemtuzumab, Golimumab, Gomiliximab, Ibalizumab, Ibritumomab, Igovomab, Imciromab, Infliximab Remicade, Inolimomab, Inotuzumab ozogamicin, Ipilimumab, Iratumumab, Keliximab, Labetuzumab, Lebrilizumab, Lemalesomab, Lerdelimumab, Lexatumumab, Libivirurnab, Lintuzumab, Lucatumumab, Lumiliximab, Mapatumumab, Maslimomab, Matuzumab, Mepolizumab, Metelimumab, Milatuzumab, Minretumomab, Mitumomab, Morolimumab, Motavizumab, Muromonab, MYO-029, Nacolomab, Naptumomab, Natalizumab, Nebacumab, Necitumumab, Nerelimomab, Nimotuzumab, Nofetumomab, Ocrelizumab, Odulimomab, Ofatumumab, Omalizumab, Oportuzumab, Oregovomab, Otelixizumab, Pagibaximab, Palivizumab, Panitumumab, Panobacumab, Pascolizumab, Pemtumomab, Pertuzumab, Pexelizumab, Pintumomab, Priliximab, Pritumumab, PRO 140, Rafivirumab, Ramucirumab, Ranibizumab, Raxibacumab, Regavirumab, Reslizumab, Rilonacept, Rituximab, Robatumumab, Rovelizumab, Rozrolimupab, Ruplizumab, Satumomab, Sevirumab, Sibrotuzumab, Siltuximab, Siplizumab, Solanezumab, Sonepcizumab, Sontuzumab, Stamulurnab, Sulesomab, Tacatuzumab, Tadocizumab, Talizumab, Tanezumab, Tapliturnomab, Tefibazumab. Telimomab, Tenatumomab, Teneliximab, Teplizumab, TGN1412, Ticilimumab (=tremelimumab), Tigatuzumab, TNX-355 (=ibalizumab), TNX-650, TNX-901 (=talizumab), Tocilizumab, Toralizumab, Tositumomab, Trastuzumab, Tremelimumab, Tucotuzumab, Tuvirumab, Urtoxazumab, Ustekinumab, Vapaliximab, Vedolizumab, Veltuzumab, Vepalimomab, Visilizumab, Volociximab, Votumumab, Zalutumumab, Zanolimumab, Ziralimumab, and Zolimomab.

In some embodiments, the antibody is adalimumab, infliximab, tocilizumab, vedolizumab, eculizumab, alemtuzumab, natalizumab, atezolizumab, bevacizumab, cetuximab, daratumumab, ipilimumab, nivolumab, obinutuzumab, pembrolizumab, pertuzumab, ramucirumab, rituximab, trastuzumab, golimumab, ustekinumab, denosumab, certolizumab pegol, secukinumab, or blinatumomab.

In certain aspects, the methods described herein comprises determining a level of an analyte in a serum of a patient. In some embodiments, the analyte comprises an anti-drug antibody or antigen-binding thereof. In some embodiments, the antibody or antigen-binding fragment thereof binds to a therapeutic antibody. Examples of the therapeutic antibody include, but are not limited to, 3F8, Abagovomab, Abatacept, Abciximab, ACZ885, Adalimumab, Adecatumumab, Afelimomab, Aflibercept, Afutuzumab, Alacizumab, Alemtuzumab, Altumomab, Anatumomab, Anrukinzumab, Apolizumab, Arcitumomab, Aselizumab, Atlizumab, Atorolimumab, Bapineuzumab, Basiliximab, Bavituximab, Bectumomab, Belatacept, Belimumab, Bertilimumab, Besilesomab, Bevacizumab, Biciromab, Bivatuzumab, Blinatumomab, Canakinumab, Cantuzumab, Capromab, Catumaxomab, Cedelizumab, Certolizumab, Cetuximab Erbitux, Citatuzumab, Cixutumumab, Clenoliximab, CNTO 1275 (=ustekinumab), CNTO 148 (=golimumab), Conatumumab, Dacetuzumab, Dacliximab (=daclizumab), Daclizumab, Denosumab, Detumomab, Dorlimomab, Dorlixizumab, Ecromeximab, Eculizumab, Edobacomab, Edrecolomab, Efalizumab, Efungumab, Elsilimomab, Enlimomab, Epitumomab, Epratuzumab, Erlizumab, Ertumaxomab, Etanercept, Etaracizumab, Exbivirumab, Fanolesomab, Faralimomab, Felvizumab, Figitumumab, Fontolizumab, Foravirumab, Galiximab, Gantenerumab, Gavilimomab, Gemtuzumab, Golimumab, Gomiliximab, Ibalizumab, Ibritumomab, Igovomab, Imciromab, Infliximab Remicade, Inolimomab, Inotuzumab ozogamicin, Ipilimumab, Iratumumab, Keliximab, Labetuzumab, Lebrilizumab, Lemalesomab, Lerdelimumab, Lexatumumab, Libivirurnab, Lintuzumab, Lucatumumab, Lumiliximab, Mapatumumab, Maslimomab, Matuzumab, Mepolizumab, Metelimumab, Milatuzumab, Minretumomab, Mitumomab, Morolimumab, Motavizumab, Muromonab, MYO-029, Nacolomab, Naptumomab, Natalizumab, Nebacumab, Necitumumab, Nerelimomab, Nimotuzumab. Nofetumomab, Ocrelizumab, Odulimomab, Ofatumumab, Omalizumab, Oportuzumab, Oregovomab, Otelixizumab, Pagibaximab, Palivizumab, Panitumumab, Panobacumab, Pascolizumab, Pemtumomab, Pertuzumab, Pexelizumab, Pintumomab, Priliximab, Pritumumab, PRO 140, Rafivirumab, Ramucirumab, Ranibizumab, Raxibacumab, Regavirumab, Reslizumab, Rilonacept, Rituximab, Robatumumab, Rovelizumab, Rozrolimupab, Ruplizumab, Satumomab, Sevirumab, Sibrotuzumab, Siltuximab, Siplizumab, Solanezumab, Sonepcizumab, Sontuzumab, Stamulurnab, Sulesomab, Tacatuzumab, Tadocizumab, Talizumab, Tanezumab, Tapliturnomab, Tefibazumab, Telimomab, Tenatumomab, Teneliximab, Teplizumab, TGN1412, Ticilimumab (=tremelimumab), Tigatuzumab, TNX-355 (=ibalizumab), TNX-650, TNX-901 (=talizumab), Tocilizumab, Toralizumab, Tositumomab, Trastuzumab, Tremelimumab, Tucotuzumab, Tuvirumab, Urtoxazumab, Ustekinumab, Vapaliximab, Vedolizumab, Veltuzumab, Vepalimomab, Visilizumab, Volociximab, Votumumab, Zalutumumab, Zanolimumab, Ziralimumab, and Zolimomab.

In some embodiments, the antibody is adalimumab, infliximab, tocilizumab, vedolizumab, eculizumab, alemtuzumab, natalizumab, atezolizumab, bevacizumab, cetuximab, daratumumab, ipilimumab, nivolumab, obinutuzumab, pembrolizumab, pertuzumab, ramucirumab, rituximab, trastuzumab, golimumab, ustekinumab, denosumab, certolizumab pegol, secukinumab, or blinatumomab.

In some embodiments, the anti-drug antibody or antigen-binding fragment thereof comprises an anti-idiotypic antibody to the therapeutic antibody. In some embodiments, the anti-idiotypic antibody comprises an antigen blocking anti-idiotype antibody, a non-blocking anti-idiotype antibody, or a complex specific anti-idiotype antibody.

The methods and devices described herein can be used in therapeutic drug monitoring (TDM) applications. Therapeutic drug monitoring is a clinical practice of measuring the level of drugs in biological fluids to make sure that the dosage of drugs administered is safe and effective. The drug concentration in biological fluids (e.g., plasma, serum, or blood) of a patient are measured at varying times during the course of treatment. This information is used to individualize dosage so that drug concentrations can be maintained within a target rage expected under the dosage administered. After analyzing the information, taking into account the patient's clinical condition, drug history, age, weight, pharmacodynamic relationship between drug concentrations and pharmacological efficacy and/or toxicity, sampling time, etc., healthcare providers (e.g., physicians, nurses, clinical pharmacists, medical laboratory scientists) can adjust the dosage of the drug. In some cases, healthcare providers increase the dosage of the drug. In some cases, healthcare providers decrease the dosage of the drug. In some cases, healthcare providers maintain the dosage of the drug. This can help improve clinical outcome by enhancing efficacy, decrease toxicity, and assisting with diagnosis.

Examples of commonly monitored drugs include, but not limited to, vancomycin, gentamycin, amikacin, digoxin, procainamide, lidocaine, phenytoin, phenobarbital, cyclosporine, tacrolimus, lithium, valproic acid, perhexiline, sirolimus, tacrolimus, thiopurines, methotrexate, Janus Kinase Inhibitors, Sphingosine-1-phosphate receptor modulators, infliximab, adalimumab, golimumab, certolizumab, (RA Ghiculescu, Aust Prescr 31:42-4 (2008); Peter M. Irving et al., Gastroenterology, 162(5): 1512-1524 (2022)). Other drugs suitable for therapeutic drug monitoring may be used.

Therapeutic drug monitoring can be used to diagnose and/or treat many different diseases and/or conditions. In some cases, the methods and devices described herein can be used to monitor antibodies and other biomarkers during multiple sclerosis (MS) and other immunosuppressant drug treatments. In some cases, the methods and devices described herein can be used to monitor drug levels against and biomarkers of disease state. In some cases, the methods and devices described herein can be used to monitor antibodies against and other biomarkers post vaccination.

Examples of the diseases and/or conditions include, but not limited to, viral infections, microbial infections, autoimmune diseases, heart diseases, inflammatory diseases, seizure, and bipolar disorder. In some cases, the diseases and/or conditions comprise corona virus, flu, monkey pox, or AIDS. In some cases, the diseases and/or conditions comprise multiple sclerosis. In some cases, the diseases and/or conditions comprise polyomavirus, JC virus, progressive multifocal leukoencephalopathy, Guillain-Barré syndrome, Myasthenia Gravis.

### Measurements of SARS-CoV-2 Antibodies

The Sars-CoV-2 (COVID-19) pandemic has highlighted the importance and accelerated acceptance of at home diagnostics across the globe. Traditionally, sample matrices such as blood, serum, plasma, or nasopharyngeal that diagnostic testing have relied on are often impractical, difficult, and/or even painful, to self-collect in an at home setting. Thus, there has been an increased interest in determining the utility and feasibility of non-invasive collections such as saliva and nasal specimens in diagnostics and correlating their biomarker levels with that found in serum.

The systemic antibody response to Sars-CoV-2 Spike RNA vaccination is detectable in saliva (Becker M, et al., Nat. Commun. 12(1), 1-8 (2021); Sheikh-Mohamed Set al., Mucosal Immunol. (January) (2022); Klingler J, et al., Front. Immunol. 12 (December), 1-12 (2021), Tu M K, et al., J. Immunol. 208(4), 819-826 (2022)) and nasal specimens (Mades A, et al., Sci. Rep. 11(1), 1-6 (2021); Chan R W Y, et al., Front. Immunol. 12 (October), 1-9 (2021); Guerrieri M, et al., Vaccines. 9(12), 1-14 (2021)), providing a good model to investigate their application for determination of serum biomarker levels. The IgG antibody response present in saliva is derived from blood by passive leakage via gingival crevicular fluid (Subbarao K C et al., J Pharm Bioallied Sci. 11 ((Suppl 2)), S135-S139 (2019); Brandtzaeg P., J. Oral Microbiol. 5(2013), 1-24 (2013)), an inflammatory exudate from periodontal tissue. There is also passive diffusion to nasal mucosa from blood (Persson C G A, et al., Scand. J. Immunol. 47, 302-313 (1998)) though there are indications that mRNA vaccines can also induce the local nasal inflammatory response (Chan R W Y, et al., Front. Immunol. 12 (October), 1-9 (2021); Guerrieri M, et al., Vaccines. 9(12), 1-14 (2021)).

An enzyme linked immunosorbent assay (ELISA) has been developed to quantitate Covid-19 specific antibodies in serum, saliva, and nasal sample matrices. In some embodiments, the ELISA assay to quantitate Covid-19 specific antibodies within the different matrices use a his-tagged SARS-CoV-2 S1 protein bound to nickel coated plates. To standardize quantitation, Sars Cov-2 antibody levels may be quantified using standards calibrated to the WHO international standard and expressed as Binding Antibody Units (BAU).

Provided herein are methods of determining a level of SARS-Cov-2 antibodies in a serum of a patient in need thereof, comprising: (a) perturbing a soft tissue in a mouth or other cavity of the patient; (b) collecting a sample of fluid from the mouth or other cavity of the patient; and (c) measuring the level of the SARS-CoV-2 antibodies in the sample of fluid from the mouth or other cavity of the patient, wherein measuring the SARS-CoV-2 antibodies in the sample of fluid from the mouth or other cavity of the patient determines the level of the SARS-CoV-2 antibodies in the serum of the patient. In some embodiments, the measuring the level of the SARS-Cov-2 antibodies in the sample of fluid from the mouth or other cavity of the patient further comprises normalizing the level of the SARS-Cov-2 antibodies to total IgG concentration in the sample of fluid from the mouth or other cavity of the patient.

In some embodiments, the soft tissue in the mouth of the patient comprises the gums. In some embodiments, the other cavity of the patient comprises a nostril. In some embodiments, the soft tissue in the mouth of the patient is perturbed for at least two minutes. In some embodiments, the soft tissue in the mouth of the patient is perturbed with a device selected from the group consisting of a manual toothbrush, an electric toothbrush, a retainer, an oral irrigator, a water flosser, a finger cot, a pic, a tongue scraper, a q-tip, and a dental floss. In some embodiments, prior to perturbing the soft tissue in a mouth of the patient, the mouth of the patient is cleaned. In some embodiments, the mouth of the patient is cleaned with a toothbrush and/or dental floss.

In some embodiment, the SARS-CoV-2 antibodies comprises SARS-CoV-2 Spike Protein S1 antibodies. In some embodiments, the SARS-CoV-2 antibodies comprises monoclonal, polyclonal, or recombinant antibodies, chimeric antibodies, humanized antibodies, and any fragments thereof. In some embodiments, the SARS-CoV-2 antibodies are rabbit, mouse, or human antibodies.

### Measurements of Natalizumab

Natalizumab is a monoclonal antibody targeting the a4 chain of integrins expressed on the surface of immune cells and required for proper cell migration to sites of infection. Several side effects, often severe, are associated with the administration of natalizumab, primarily because of its weakening effect on the immune system. Such side effects include an increased risk of developing various infections, such as urinary tract infection, lower respiratory tract infection, vaginitis, and brain infection including Herpes-mediated encephalitis and meningitis, and progressive multifocal leukoencephalopathy (PML). The risk of developing PML is one of the most studied adverse events associated with the utilization of natalizumab in the treatment of multiple sclerosis because of its severity, unpredictability, and incurable nature. The etiologic agent is the John Cunningham polyomavirus (JCV). Testing positive for antibodies against JCV serves as a primary risk stratification tool in the clinical use of natalizumab to better assess the risk of developing PML (Plavina, T. et al., Ann Neurol. 76(6). 802-812 (2014)). Secondary risk stratification markers would greatly aid in the clinical management of patients receiving natalizumab, not only to mitigate the risk of developing PML, but also to mitigate the risk of suffering from other types of infections which are more frequently observed in the patient population than PML. It has been shown that extended interval dosing (EID) of natalizumab patients beyond the approved 4-week dosing decreases risk of PML by 94% (Ryerson, L Z. et al., Neurology. 93(15) e1452-e1462 (2019)). Recent studies have shown that every six-week natalizumab IV administration provides a high level of efficacy in controlling MS disease activity in patients who switched from the approved every four-week dosing regimen while also being associated with an 88 percent reduction in the probability of developing PML (Foley John F, et al., Lancet 21(7), 608-619 (2022)).

Natalizumab, is an IgG4 that is capable of in vivo arm exchange (Labrijn, Aran F et al., Nature Biotechnology 27(8): 767-71 (2009)), where a heavy chain and associated light chain from one antibody is swapped with that from another. The extent of arm exchange may depend on both the concentration of natalizumab and the total IgG4 concentration present in circulation. Bivalent natalizumab has ˜20-fold increased affinity for binding to T lymphoblastoid cells when compared to monovalent suggesting that it binds bivalently to a4b1 integrin (Yu, Yamei et al., Journal of Biological Chemistry 288(45): 32314-25 (2013)). Functional declines in receptor occupancy as drug levels reach trough may contribute to limited immune cell trafficking to the CNS and provide some surveillance of JCV, lowering risk.

This differences in affinity between bivalent and monovalent natalizumab is expected to lead to corresponding differences in receptor occupancy and thus the degree to which immune surveillance of the brain occurs. Therefore, the evaluation of natalizumab forms in patient serum is important to undertake to discern if their levels, as well as overall natalizumab concentration, contribute to adverse events. If natalizumab serum levels can be accurately predicted from a non-invasive sample such as saliva, this would add value to current monitoring, allowing self-collection and testing to occur before the patient is due for infusion- and will potentially enable physicians to determine if treatment should go ahead on the standard treatment regime or be delayed.

Provided herein are methods of determining a level of natalizumab in a serum of a patient in need thereof, comprising: (a) perturbing a soft tissue in a mouth or other cavity of the patient; (b) collecting a sample of fluid from the mouth or other cavity of the patient; and (c) measuring the level of natalizumab in the sample of fluid from the mouth or other cavity of the patient, wherein measuring natalizumab in the sample of fluid from the mouth or other cavity of the patient determines the level of natalizumab in the serum of the patient. In some embodiments, the measuring the level of natalizumab in the sample of fluid from the mouth or other cavity of the patient further comprises normalizing the level of natalizumab (total, bivalent, and/or monovalent) to total IgG concentration in the sample of fluid from the mouth or other cavity of the patient. In some embodiments, IgG4 levels are also measured and normalized with total IgG from the sample of fluid from the mouth or other cavity of the patient.

In some embodiments, the soft tissue in the mouth of the patient comprises the gums. In some embodiments, the other cavity of the patient comprises a nostril. In some embodiments, the soft tissue in the mouth of the patient is perturbed for 0-10 seconds, 10-30 seconds, 30 seconds-1 minute, or 1-2 minutes. In some embodiments, the soft tissue in the mouth of the patient is perturbed with a device selected from the group consisting of a manual toothbrush, an electric toothbrush, a retainer, an oral irrigator, a water flosser, a finger cot, and a dental floss. In some embodiments, prior to perturbing the soft tissue in a mouth of the patient, the mouth of the patient is cleaned. In some embodiments, the mouth of the patient is cleaned with a toothbrush and/or dental floss.

### Normalization of an Analyte Level to Total IgG Level

In some embodiments, the methods described herein further comprise measuring the level of the analyte in the sample of fluid from the mouth or other cavity of the patient determines the level of the analyte in the serum of the patient. In some embodiments, the level of the analyte in a sample is normalized to total IgG concentration present in the sample. In some embodiments, the level of the analyte in a sample is measured first. In some embodiments, the total IgG level in a sample is measured first. In some embodiments, the level of the analyte and/or the total IgG level is measured using ELISA. In some embodiments, the level of the analyte in a sample is normalized and is expressed as the level of the analyte per IgG (ug/ml). In some embodiments, an average total serum IgG concentration is determined by calculating the mean IgG of all the serum donated. In some embodiments, the mean serum IgG concentration is used to predict the serum level of the analyte in the sample.

In some embodiments, the level of the analyte in a sample and/or the total IgG level is measured before vaccination. In some embodiments, the level of the analyte in a sample and/or the total IgG level is measured after vaccination. In some embodiments, the level of the analyte in a sample and/or the total IgG level is measured before treatment. In some embodiments, the level of the analyte in a sample and/or the total IgG level is measured during treatment. In some embodiments, the level of the analyte in a sample and/or the total IgG level is measured after treatment. In some embodiments, the level of the analyte in a sample and/or the total IgG level is measured before the subject has a symptom and/or condition of a disease. In some embodiments, the level of the analyte in a sample and/or the total IgG level is measured during the subject has a symptom and/or condition of a disease. In some embodiments, the level of the analyte in a sample and/or the total IgG level is measured after the subject has a symptom and/or condition of a disease. In some embodiments, the disease or condition comprises virus infection, bacterial infection, autoimmune disease, corona virus, multiple sclerosis, polyomavirus, JC virus, progressive multifocal leukoencephalopathy, Guillain-Barré syndrome, Myasthenia Gravis, flu, monkey pox, AIDS or a combination thereof.

In some embodiments, the normalized level of the analyte is used to monitor antibodies against and other biomarkers during immunosuppressant drug treatment. In some embodiments, the normalized level of the analyte is used to monitor drug levels against and other biomarkers of disease state. In some embodiments, the normalized level of the analyte is used to monitor antibodies against and other biomarkers post vaccination.

In some embodiments, the normalized level of the analyte is used to monitor antibodies against and other biomarkers before, during, or after immunosuppressant drug treatment. In some embodiments, the immunosuppressant drug treatment is applied to treat an autoimmune disease. In some embodiment, the autoimmune disease comprises multiple sclerosis. In some embodiments, the antibody comprises a monoclonal antibody, monovalent antibody, an intact antibody, a bivalent antibody, a scrambled antibody, a total antibody, a polyclonal antibody, a recombinant antibody, a chimeric antibody, a humanized antibody, a camelid antibody, a CDR-grafted antibody, F(ab)2, Fv, scFv, IgGΔCH2, F(ab′)2, scFv2CH3, F(ab), VL, VH, scFv4, scFv3, scFv2, dsFv, Fv, scFv-Fc, (scFv)2, a disulfide linked Fv, a single domain antibody (dAb), a diabody, a bispecific antibody, a multispecific antibody, a dual specific antibody, an anti-idiotypic antibody, a nanobody, a VHH antibody, IgA, IgD, IgE, IgG, IgM, a modified antibody, non-depleting IgG antibodies, T-bodies, Fc or Fab variants thereof. In some embodiments, the antibody is a mouse antibody or a human antibody.

In some embodiments, the antibody or antigen-binding fragment thereof described herein specifically binds to a SARS-CoV-2 spike protein. In some embodiments, the antibody or antigen-binding fragment thereof binds to S1 domain of the SARS-CoV-2 spike protein. In some embodiments, the antibody or antigen-binding fragment thereof binds to S2 domain of the SARS-CoV-2 spike protein.

In some embodiments, the antibody or antigen-binding fragment thereof described herein specifically binds to an Fc receptor. In some embodiments, the Fc receptor is an IgG Fc receptor. In some embodiments, the Fc receptor is an IgA Fc receptor. In some embodiments, the Fc receptor is an IgE Fc receptor. In some embodiments, the Fc receptor is an IgM Fc receptor.

In some embodiments, the IgG Fc receptor is a neonatal Fc receptor (FcRn). The FcRN plays a key role in regulating the life cycle of IgG and serum albumin. In some cases, FcRn extends the serum half-life of therapeutic antibodies. In some cases, FcRn extends the half-life of pathogenic IgG antibodies and promote autoimmune diseases. The antibodies or Fc fragments that have high binding affinity with FcRn can be used to treat autoimmune diseases (e.g. myasthenia gravis) by disrupting the binding of FcRn to pathogenic IgG antibodies.

In some embodiments, the antibody or antigen-binding fragment described herein comprises adalimumab, infliximab, tocilizumab, vedolizumab, eculizumab, alemtuzumab, natalizumab, atezolizumab, bevacizumab, cetuximab, daratumumab, ipilimumab, nivolumab, obinutuzumab, pembrolizumab, pertuzumab, ramucirumab, rituximab, trastuzumab, golimumab, ustekinumab, denosumab, certolizumab pegol, secukinumab, alemtuzumab, or blinatumomab. In some embodiments, the antibody or antigen-binding fragment described herein comprises alemtuzumab. In some embodiments, the antibody or antigen-binding fragment described herein comprises natalizumab.

In some embodiments, the antibody or antigen binding fragment described herein comprises a monoclonal antibody. Examples of monoclonal antibodies include, but are not limited to 3F8, Abagovomab, Abatacept, Abciximab, AC885, Adalimumab, Adecatumumab, Afelimomab, Aflibercept, Afutuzumab, Alacizumab, Alemtuzumab, Altumomab, Anatumomab, Anrukinzumab, Apolizumab, Arcitumomab, Aselizumab, Atlizumab, Atorolimumab, Bapineuzumab, Basiliximab, Bavituximab, Bectumomab, Belatacept, Belimumab, Bertilimumab, Besilesomab, Bevacizumab, Biciromab, Bivatuzumab, Blinatumomab, Canakinumab, Cantuzumab, Capromab, Catumaxomab, Cedelizumab, Certolizumab, Cetuximab Erbitux, Citatuzumab, Cixutumumab, Clenoliximab, CNTO 1275 (=ustekinumab), CNTO 148 (=golimumab), Conatumumab, Dacetuzumab, Dacliximab (=daclizumab), Daclizumab, Denosumab, Detumomab, Dorlimomab, Dorlixizumab, Ecromeximab, Eculizumab, Edobacomab, Edrecolomab, Efalizumab, Efungumab, Elsilimomab, Enlimomab, Epitumomab, Epratuzumab, Erlizumab, Ertumaxomab, Etanercept, Etaracizumab, Exbivirumab, Fanolesomab, Faralimomab, Felvizumab, Figitumumab, Fontolizumab, Foravirumab, Galiximab, Gantenerumab, Gavilimomab, Gemtuzumab, Golimumab, Gomiliximab, Ibalizumab, Ibritumomab, Igovomab, Imciromab, Infliximab Remicade, Inolimomab, Inotuzumab ozogamicin, Ipilimumab, Iratumumab, Keliximab, Labetuzumab, Lebrilizumab, Lemalesomab, Lerdelimumab, Lexatumumab, Libivirurnab, Lintuzumab, Lucatumumab, Lumiliximab, Mapatumumab, Maslimomab, Matuzumab, Mepolizumab, Metelimumab, Milatuzumab, Minretumomab, Mitumomab, Morolimumab, Motavizumab, Muromonab, MYO-029, Nacolomab, Naptumomab, Natalizumab, Nebacumab, Necitumumab, Nerelimomab, Nimotuzumab, Nofetumomab, Ocrelizumab, Odulimomab, Ofatumumab, Omalizumab, Oportuzumab, Oregovomab, Otelixizumab, Pagibaximab, Palivizumab, Panitumumab, Panobacumab, Pascolizumab, Pemtumomab, Pertuzumab, Pexelizumab, Pintumomab, Priliximab, Pritumumab, PRO 140, Rafivirumab, Ramucirumab, Ranibizumab, Raxibacumab, Regavirumab, Reslizumab, Rilonacept, Rituximab, Robatumumab, Rovelizumab, Rozrolimupab, Ruplizumab, Satumomab, Sevirumab, Sibrotuzumab, Siltuximab, Siplizumab, Solanezumab, Sonepcizumab, Sontuzumab, Stamulurnab, Sulesomab, Tacatuzumab, Tadocizumab, Talizumab, Tanezumab, Tapliturnomab, Tefibazumab, Telimomab, Tenatumomab, Teneliximab, Teplizumab, TGN1412, Ticilimumab (=tremelimumab), Tigatuzumab, TNX-355 (=ibalizumab), TNX-650, TNX-901 (=talizumab), Tocilizumab, Toralizumab, Tositumomab, Trastuzumab, Tremelimumab, Tucotuzumab, Tuvirumab, Urtoxazumab, Ustekinumab, Vapaliximab, Vedolizumab, Veltuzumab, Vepalimomab, Visilizumab, Volociximab, Votumumab, Zalutumumab, Zanolimumab, Ziralimumab, and Zolimomab.

In some embodiments, the antibody is adalimumab, infliximab, tocilizumab, vedolizumab, eculizumab, alemtuzumab, natalizumab, atezolizumab, bevacizumab, cetuximab, daratumumab, ipilimumab, nivolumab, obinutuzumab, pembrolizumab, pertuzumab, ramucirumab, rituximab, trastuzumab, golimumab, ustekinumab, denosumab, certolizumab pegol, secukinumab, or blinatumomab.

In some embodiments, the normalized level of the analyte is used to monitor anti-drug antibodies before, during, or after treatment. In some embodiments, the analyte comprises an anti-drug antibody or antigen-binding thereof. In some embodiments, the anti-drug antibody or antigen-binding fragment thereof binds to a therapeutic antibody. In some embodiments, the anti-drug antibody or antigen-binding fragment thereof comprises an anti-idiotypic antibody to the therapeutic antibody. In some embodiments, the anti-idiotypic antibody comprises an antigen blocking anti-idiotype antibody, a non-blocking anti-idiotype antibody, or a complex specific anti-idiotype antibody.

Examples of the therapeutic antibody include, but are not limited to, 3F8, Abagovomab, Abatacept, Abciximab, ACZ885, Adalimumab, Adecatumumab, Afelimomab, Aflibercept, Afutuzumab, Alacizumab, Alemtuzumab, Altumomab, Anatumomab, Anrukinzumab, Apolizumab, Arcitumomab, Aselizumab, Atlizumab, Atorolimumab, Bapineuzumab, Basiliximab, Bavituximab, Bectumomab, Belatacept, Belimumab, Bertilimumab, Besilesomab, Bevacizumab, Biciromab, Bivatuzumab, Blinatumomab, Canakinumab, Cantuzumab, Capromab, Catumaxomab, Cedelizumab, Certolizumab, Cetuximab Erbitux, Citatuzumab, Cixutumumab, Clenoliximab, CNTO 1275 (=ustekinumab), CNTO 148 (=golimumab), Conatumumab, Dacetuzumab, Dacliximab (=daclizumab), Daclizumab, Denosumab, Detumomab, Dorlimomab, Dorlixizumab, Ecromeximab, Eculizumab, Edobacomab, Edrecolomab, Efalizumab, Efungumab, Elsilimomab, Enlimomab, Epitumomab, Epratuzumab, Erlizumab, Ertumaxomab, Etanercept, Etaracizumab, Exbivirumab, Fanolesomab, Faralimomab, Felvizumab, Figitumumab, Fontolizumab, Foravirumab, Galiximab, Gantenerumab, Gavilimomab, Gemtuzumab, Golimumab, Gomiliximab, Ibalizumab, Ibritumomab, Igovomab, Imciromab, Infliximab Remicade, Inolimomab, Inotuzumab ozogamicin, Ipilimumab, Iratumumab, Keliximab, Labetuzumab, Lebrilizumab, Lemalesomab, Lerdelimumab, Lexatumumab, Libivirurnab, Lintuzumab, Lucatumumab, Lumiliximab, Mapatumumab, Maslimomab, Matuzumab, Mepolizumab, Metelimumab, Milatuzumab, Minretumomab, Mitumomab, Morolimumab, Motavizumab, Muromonab, MYO-029, Nacolomab, Naptumomab, Natalizumab, Nebacumab, Necitumumab, Nerelimomab, Nimotuzumab, Nofetumomab, Ocrelizumab, Odulimomab, Ofatumumab, Omalizumab, Oportuzumab, Oregovomab, Otelixizumab, Pagibaximab, Palivizumab, Panitumumab, Panobacumab, Pascolizumab, Pemtumomab, Pertuzumab, Pexelizumab, Pintumomab, Priliximab, Pritumumab, PRO 140, Rafivirumab, Ramucirumab, Ranibizumab, Raxibacumab, Regavirumab, Reslizumab, Rilonacept, Rituximab, Robatumumab, Rovelizumab, Rozrolimupab, Ruplizumab, Satumomab, Sevirumab, Sibrotuzumab, Siltuximab, Siplizumab, Solanezumab, Sonepcizumab, Sontuzumab, Stamulurnab, Sulesomab, Tacatuzumab, Tadocizumab, Talizumab, Tanezumab, Tapliturnomab, Tefibazumab, Telimomab, Tenatumomab, Teneliximab, Teplizumab, TGN1412, Ticilimumab (=tremelimumab), Tigatuzumab, TNX-355 (=ibalizumab), TNX-650, TNX-901 (=talizumab), Tocilizumab, Toralizumab, Tositumomab, Trastuzumab, Tremelimumab, Tucotuzumab, Tuvirumab, Urtoxazumab, Ustekinumab, Vapaliximab, Vedolizumab, Veltuzumab, Vepalimomab, Visilizumab, Volociximab, Votumumab, Zalutumumab, Zanolimumab, Ziralimumab, and Zolimomab.

In some embodiments, the antibody is adalimumab, infliximab, tocilizumab, vedolizumab, eculizumab, alemtuzumab, natalizumab, atezolizumab, bevacizumab, cetuximab, daratumumab, ipilimumab, nivolumab, obinutuzumab, pembrolizumab, pertuzumab, ramucirumab, rituximab, trastuzumab, golimumab, ustekinumab, denosumab, certolizumab pegol, secukinumab, or blinatumomab.

### Devices for Determining a Level of a Serum Analyte

Provided herein are devices for determining a level of an analyte in the serum of a patient in need thereof. In some embodiments, the device comprises (a) an oral care tool or a part thereof and (b) a lateral flow assay test. In some embodiments, the oral care tool comprises a manual toothbrush, an electric toothbrush, a retainer, an oral irrigator, a water flosser, a finger cot, a dental floss, a gum stimulator, a swab, a dental sponge, a swab stick, a form tip, an interdental brush, a dental scraper, a dental scaler, a dental pick, a dental stick, or a combination thereof.

The methods described herein comprises determining a level of an analyte in a serum of a patient Examples of analytes include, but not limited to, an antibody or antigen-binding fragment thereof, biomarker, DNA, RNA, hormone, lipid, drug, anti-drug antibody or antigen-binding fragment thereof, other naturally occurring molecule or a combination thereof. In some embodiments, the analyte is an antibody. In some embodiments, the antibody is a monoclonal antibody, monovalent antibody, an intact antibody, a bivalent antibody, a scrambled antibody, a total antibody, a polyclonal antibody, a recombinant antibody, a chimeric antibody, a humanized antibody, a camelid antibody, a CDR-grafted antibody, F(ab)2, Fv, scFv, IgGΔCH2, F(ab′)2, scFv2CH3, F(ab), VL, VH, scFv4, scFv3, scFv2, dsFv, Fv, scFv-Fc, (scFv)2, a disulfide linked Fv, a single domain antibody (dAb), a diabody, a bispecific antibody, a multispecific antibody, a dual specific antibody, an anti-idiotypic antibody, a nanobody, a VHH antibody, IgA, IgD, IgE, IgG, IgM, a modified antibody, non-depleting IgG antibodies, T-bodies, Fc or Fab variants thereof. In some embodiments, the antibody is a mouse antibody or a human antibody.

In some embodiments, the antibody or antigen-binding fragment thereof described herein specifically binds to a SARS-CoV-2 spike protein. In some embodiments, the antibody or antigen-binding fragment thereof binds to S1 domain of the SARS-CoV-2 spike protein. In some embodiments, the antibody or antigen-binding fragment thereof binds to S2 domain of the SARS-CoV-2 spike protein.

In some embodiments, the antibody or antigen-binding fragment thereof described herein specifically binds to an Fc receptor. In some embodiments, the Fc receptor is an IgG Fc receptor. In some embodiments, the Fc receptor is an IgA Fc receptor. In some embodiments, the Fc receptor is an IgE Fc receptor. In some embodiments, the Fc receptor is an IgM Fc receptor.

In some embodiments, the IgG Fc receptor is a neonatal Fc receptor (FcRn). The FcRN plays a key role in regulating the life cycle of IgG and serum albumin. In some cases, FcRn extends the serum half-life of therapeutic antibodies. In some cases, FcRn extends the half-life of pathogenic IgG antibodies and promote autoimmune diseases. The antibodies or Fc fragments that have high binding affinity with FcRn can be used to treat autoimmune diseases (e.g. myasthenia gravis) by disrupting the binding of FcRn to pathogenic IgG antibodies.

In some embodiments, the antibody or antigen-binding fragment described herein comprises adalimumab, infliximab, tocilizumab, vedolizumab, eculizumab, alemtuzumab, natalizumab, atezolizumab, bevacizumab, cetuximab, daratumumab, ipilimumab, nivolumab, obinutuzumab, pembrolizumab, pertuzumab, ramucirumab, rituximab, trastuzumab, golimumab, ustekinumab, denosumab, certolizumab pegol, secukinumab, alemtuzumab, or blinatumomab. In some embodiments, the antibody or antigen-binding fragment described herein comprises alemtuzumab. In some embodiments, the antibody or antigen-binding fragment described herein comprises natalizumab.

In some embodiments, the antibody or antigen binding fragment described herein comprises a monoclonal antibody. Examples of monoclonal antibodies include, but are not limited to 3F8, Abagovomab, Abatacept, Abciximab, ACZ885, Adalimumab, Adecatumumab, Afelimomab, Aflibercept, Afutuzumab, Alacizumab, Alemtuzumab, Altumomab, Anatumomab, Anrukinzumab, Apolizumab, Arcitumomab, Aselizumab, Atlizumab, Atorolimumab, Bapineuzumab, Basiliximab, Bavituximab, Bectumomab, Belatacept, Belimumab, Bertilimumab, Besilesomab, Bevacizumab, Biciromab, Bivatuzumab, Blinatumomab, Canakinumab, Cantuzumab, Capromab, Catumaxomab, Cedelizumab, Certolizumab, Cetuximab Erbitux, Citatuzumab, Cixutumumab, Clenoliximab, CNTO 1275 (=ustekinumab), CNTO 148 (=golimumab), Conatumumab, Dacetuzumab, Dacliximab (=daclizumab), Daclizumab, Denosumab, Detumomab, Dorlimomab, Dorlixizumab, Ecromeximab, Eculizumab, Edobacomab, Edrecolomab, Efalizumab, Efungumab, Elsilimomab, Enlimomab, Epitumomab, Epratuzumab, Erlizumab, Ertumaxomab, Etanercept, Etaracizumab, Exbivirumab, Fanolesomab, Faralimomab, Felvizumab, Figitumumab, Fontolizumab, Foravirumab, Galiximab, Gantenerumab, Gavilimomab, Gemtuzumab, Golimumab, Gomiliximab, Ibalizumab, Ibritumomab, Igovomab, Imciromab, Infliximab Remicade, Inolimomab, Inotuzumab ozogamicin, Ipilimumab, Iratumumab, Keliximab, Labetuzumab, Lebrilizumab, Lemalesomab, Lerdelimumab, Lexatumumab, Libivirurnab, Lintuzumab, Lucatumumab, Lumiliximab, Mapatumumab, Maslimomab, Matuzumab, Mepolizumab, Metelimumab, Milatuzumab, Minretumomab, Mitumomab, Morolimumab, Motavizumab, Muromonab, MYO-029, Nacolomab, Naptumomab, Natalizumab, Nebacumab, Necitumumab, Nerelimomab, Nimotuzumab, Nofetumomab, Ocrelizumab, Odulimomab, Ofatumumab, Omalizumab, Oportuzumab, Oregovomab, Otelixizumab, Pagibaximab, Palivizumab, Panitumumab, Panobacumab, Pascolizumab, Pemtumomab, Pertuzumab, Pexelizumab, Pintumomab, Priliximab, Pritumumab, PRO 140, Rafivirumab, Ramucirumab, Ranibizumab, Raxibacumab, Regavirumab, Reslizumab, Rilonacept, Rituximab, Robatumumab, Rovelizumab, Rozrolimupab, Ruplizumab, Satumomab, Sevirumab, Sibrotuzumab, Siltuximab, Siplizumab, Solanezumab, Sonepcizumab, Sontuzumab, Stamulurnab, Sulesomab, Tacatuzumab, Tadocizumab, Talizumab, Tanezumab, Tapliturnomab, Tefibazumab, Telimomab, Tenatumomab, Teneliximab, Teplizumab, TGN1412, Ticilimumab (=tremelimumab), Tigatuzumab, TNX-355 (=ibalizumab), TNX-650, TNX-901 (=talizumab), Tocilizumab, Toralizumab, Tositumomab, Trastuzumab, Tremelimumab, Tucotuzumab, Tuvirumab, Urtoxazumab, Ustekinumab, Vapaliximab, Vedolizumab, Veltuzumab, Vepalimomab, Visilizumab, Volociximab, Votumumab, Zalutumumab, Zanolimumab, Ziralimumab, and Zolimomab.

In some embodiments, the antibody is adalimumab, infliximab, tocilizumab, vedolizumab, eculizumab, alemtuzumab, natalizumab, atezolizumab, bevacizumab, cetuximab, daratumumab, ipilimumab, nivolumab, obinutuzumab, pembrolizumab, pertuzumab, ramucirumab, rituximab, trastuzumab, golimumab, ustekinumab, denosumab, certolizumab pegol, secukinumab, or blinatumomab.

In certain aspects, the methods described herein comprises determining a level of an analyte in a serum of a patient. In some embodiments, the analyte comprises an anti-drug antibody or antigen-binding thereof. In some embodiments, the antibody or antigen-binding fragment thereof binds to a therapeutic antibody. Examples of the therapeutic antibody include, but are not limited to, 3F8, Abagovomab, Abatacept, Abciximab, ACZ885, Adalimumab, Adecatumumab, Afelimomab, Aflibercept, Afutuzumab, Alacizumab, Alemtuzumab, Altumomab, Anatumomab, Anrukinzumab, Apolizumab, Arcitumomab, Aselizumab, Atlizumab, Atorolimumab, Bapineuzumab, Basiliximab, Bavituximab, Bectumomab, Belatacept, Belimumab, Bertilimumab, Besilesomab, Bevacizumab, Biciromab, Bivatuzumab, Blinatumomab, Canakinumab, Cantuzumab, Capromab, Catumaxomab, Cedelizumab, Certolizumab, Cetuximab Erbitux, Citatuzumab, Cixutumumab, Clenoliximab, CNTO 1275 (=ustekinumab), CNTO 148 (=golimumab), Conatumumab, Dacetuzumab, Dacliximab (=daclizumab), Daclizumab, Denosumab, Detumomab, Dorlimomab, Dorlixizumab, Ecromeximab, Eculizumab, Edobacomab, Edrecolomab, Efalizumab, Efungumab, Elsilimomab, Enlimomab, Epitumomab, Epratuzumab, Erlizumab, Ertumaxomab, Etanercept, Etaracizumab, Exbivirumab, Fanolesomab, Faralimomab, Felvizumab, Figitumumab, Fontolizumab, Foravirumab, Galiximab, Gantenerumab, Gavilimomab, Gemtuzumab, Golimumab, Gomiliximab, Ibalizumab, Ibritumomab, Igovomab, Imciromab, Infliximab Remicade, Inolimomab, Inotuzumab ozogamicin, Ipilimumab, Iratumumab, Keliximab, Labetuzumab, Lebrilizumab, Lemalesomab, Lerdelimumab, Lexatumumab, Libivirurnab, Lintuzumab, Lucatumumab, Lumiliximab, Mapatumumab, Maslimomab, Matuzumab, Mepolizumab, Metelimumab, Milatuzumab, Minretumomab, Mitumomab, Morolimumab, Motavizumab, Muromonab, MYO-029, Nacolomab, Naptumomab, Natalizumab, Nebacumab, Necitumumab, Nerelimomab, Nimotuzumab, Nofetumomab, Ocrelizumab, Odulimomab, Ofatumumab, Omalizumab, Oportuzumab, Oregovomab, Otelixizumab, Pagibaximab, Palivizumab, Panitumumab, Panobacumab, Pascolizumab, Pemtumomab, Pertuzumab, Pexelizumab, Pintumomab, Priliximab, Pritumumab, PRO 140, Rafivirumab, Ramucirumab, Ranibizumab, Raxibacumab, Regavirumab, Reslizumab, Rilonacept, Rituximab, Robatumumab, Rovelizumab, Rozrolimupab, Ruplizumab, Satumomab, Sevirumab, Sibrotuzumab, Siltuximab, Siplizumab, Solanezumab, Sonepcizumab, Sontuzumab, Stamulurnab, Sulesomab, Tacatuzumab, Tadocizumab, Talizumab, Tanezumab, Tapliturnomab, Tefibazumab, Telimomab, Tenatumomab, Teneliximab, Teplizumab, TGN1412, Ticilimumab (=tremelimumab), Tigatuzumab, TNX-355 (=ibalizumab), TNX-650, TNX-901 (=talizumab), Tocilizumab, Toralizumab, Tositumomab, Trastuzumab, Tremelimumab, Tucotuzumab, Tuvirumab, Urtoxazumab, Ustekinumab, Vapaliximab, Vedolizumab, Veltuzumab, Vepalimomab, Visilizumab, Volociximab, Votumumab, Zalutumumab, Zanolimumab, Ziralimumab, and Zolimomab.

In some embodiments, the antibody is adalimumab, infliximab, tocilizumab, vedolizumab, eculizumab, alemtuzumab, natalizumab, atezolizumab, bevacizumab, cetuximab, daratumumab, ipilimumab, nivolumab, obinutuzumab, pembrolizumab, pertuzumab, ramucirumab, rituximab, trastuzumab, golimumab, ustekinumab, denosumab, certolizumab pegol, secukinumab, or blinatumomab.

In some embodiments, the anti-drug antibody or antigen-binding fragment thereof comprises an anti-idiotypic antibody to the therapeutic antibody. In some embodiments, the anti-idiotypic antibody comprises an antigen blocking anti-idiotype antibody, a non-blocking anti-idiotype antibody, or a complex specific anti-idiotype antibody.

Also disclosed herein are devices for determining the level of a serum analyte in a biological matrix using a collection device. The collection device may be used for animals, including humans. In some embodiments, the biological matrix is selected from saliva, nasal, mucosal, vaginal, and fecal samples. In some embodiments, the device can comprise stimulating features, e.g., bristles, knob, nubs, bombs, and/or flossers. Features can also be included that stimulate the tissue in and around the collection area in order to produce more sample or a sample of better quality for collection. In some embodiments, energy assistance (e.g., vibration or ultrasound) is employed to assist in the stimulation, and/or aspiration may be employed to draw the sample in. In some embodiments, affinity capture on surface of a swab, bristles, or other stimulating surface to capture either desired analytes or remove undesired components is included. In some embodiments, non-specific or blotting type surface to capture desired sample components (e.g., globulin proteins) or to remove undesired components that may interfere with the analysis (e.g. cellular debris, proteases) are included. In some embodiments, capillaries for capturing the sample via capillary action are included in the device. In some embodiments, collection features for capturing biological fluid and transferring them to either storage containers or directly to a sensor or diagnostic device are included. In some embodiments, sample collection is done into cartridges, vials, tubes, and similar containers. In some embodiments, the device comprises sensors that are electrochemical, capitative, optical, surface acoustic wave, and/or magnetic. In some embodiments, the diagnostic devices disclosed herein comprise lateral flow assays (LFA), polymerase chain reaction (PCR) assays, and/or enzyme-linked immunoassay (ELISA). In some embodiments, the device comprises wireless connectivity for device to relay information to the cloud, computer, smart phone via wifi, and/or bluetooth. In some embodiments, the devices disclosed herein are plug-in or battery powered, such as with a rechargeable or disposable battery. In some embodiments, the devices comprise an on/off switch. The analytes tested by the methods and devices disclosed herein include sample matrix components depending on sample type; blood and plasma components; and drugs, hormones, biomarkers, and/or microbes.

The devices disclosed herein include uses as point of care diagnostics, including when a patient comes into an office and either collects the sample themselves or the sample is collected by a technician. The sample can then be analyzed and interpreted by a technician, nurse, doctor, or other professional. The devices disclosed herein may also be used at home, including when the patient uses the device at home to collect the sample and sends it in for analysis, or when the patient uses the device at home to collect and analyze the sample (and includes when the patient interprets the results themselves). Additionally, the patient can use the device at home to collect and analyze the sample and the device uploads data regarding sample collection, time, date, sample type, etc. to the cloud. Data is interpreted by a remote expert such as a nurse, doctor, technician and patient is advised regarding to the result.

### Method of Treatment

In one aspect, provided herein are methods for treating a disease or condition in a subject. In some embodiments, the methods comprises (a) determining a level of an analyte in a serum of the patient, comprising: (i) perturbing a soft tissue in a mouth or other cavity of the patient; (ii) collecting a sample of fluid from the mouth or other cavity of the patient; and (iii) measuring the level of the analyte in the sample of fluid from the mouth or other cavity of the patient. In some embodiments, measuring the level of the analyte in the sample of fluid from the mouth or other cavity of the patient determines the level of the analyte in the serum of the patient. In some embodiments, the method comprises administering a drug to the subject.

In some embodiments, the method described herein further describes adjusting a dosage of the drug after an initial administration of the drug. In some embodiments, the adjusting a dosage of the drug comprises increasing, decreasing, or maintaining the dosage of the drug. In some embodiments, the adjusting a dosage of the drug comprises increasing the dosage of the drug. In some embodiments, the adjusting a dosage of the drug comprises decreasing the dosage of the drug. In some embodiments, the adjusting a dosage of the drug comprises maintaining the dosage of the drug.

In some embodiments, the disease or condition comprises virus infection, bacterial infection, autoimmune disease, corona virus, multiple sclerosis, polyomavirus, JC virus, progressive multifocal leukoencephalopathy, Guillain-Barré syndrome, Myasthenia Gravis, flu, monkey pox, AIDS or a combination thereof.

In some embodiment, the analyte comprises an antibody or antigen-binding fragment thereof, biomarker, DNA, RNA, hormone, lipid, drug, anti-drug antibody or antigen-binding fragment thereof, other naturally occurring molecule, or a combination thereof. In some embodiments, the antibody or antigen-binding fragment thereof comprises a monoclonal antibody (mAb), a monovalent antibody, an intact antibody, a bivalent antibody, a scrambled antibody, a total antibody, a polyclonal antibody, a recombinant antibody, a chimeric antibody, a humanized antibody, a camelid antibody, a CDR-grafted antibody, F(ab)2, Fv, scFv, IgGΔCH2, F(ab′)2, scFv2CH3, F(ab), VL, VH, scFv4, scFv3, scFv2, dsFv, Fv, scFv-Fc, (scFv)2, a disulfide linked Fv, a single domain antibody (dAb), a diabody, a bispecific antibody, a multispecific antibody, a dual specific antibody, an anti-idiotypic antibody, a nanobody, a VHH antibody, IgA, IgD, IgE, IgG, IgM, a modified antibody, non-depleting IgG antibodies, T-bodies, Fc or Fab variants thereof.

In some embodiments, the antibody or antigen-binding fragment thereof described herein specifically binds to a SARS-Cov-2 spike protein. In some embodiments, the antibody or antigen-binding fragment thereof binds to S1 domain of the SARS-CoV-2 spike protein. In some embodiments, the antibody or antigen-binding fragment thereof binds to S2 domain of the SARS-CoV-2 spike protein.

In some embodiments, the antibody or antigen-binding fragment thereof described herein specifically binds to an Fc receptor. In some embodiments, the Fc receptor is an IgG Fc receptor. In some embodiments, the Fc receptor is an IgA Fc receptor. In some embodiments, the Fc receptor is an IgE Fc receptor. In some embodiments, the Fc receptor is an IgM Fc receptor.

In some embodiments, the IgG Fc receptor is a neonatal Fc receptor (FcRn). The FcRN plays a key role in regulating the life cycle of IgG and serum albumin. In some cases, FcRn extends the serum half-life of therapeutic antibodies. In some cases, FcRn extends the half-life of pathogenic IgG antibodies and promote autoimmune diseases. The antibodies or Fc fragments that have high binding affinity with FcRn can be used to treat autoimmune diseases (e.g. myasthenia gravis) by disrupting the binding of FcRn to pathogenic IgG antibodies.

In some embodiments, the antibody or antigen-binding fragment described herein comprises adalimumab, infliximab, tocilizumab, vedolizumab, eculizumab, alemtuzumab, natalizumab, atezolizumab, bevacizumab, cetuximab, daratumumab, ipilimumab, nivolumab, obinutuzumab, pembrolizumab, pertuzumab, ramucirumab, rituximab, trastuzumab, golimumab, ustekinumab, denosumab, certolizumab pegol, secukinumab, alemtuzumab, or blinatumomab. In some embodiments, the antibody or antigen-binding fragment described herein comprises alemtuzumab. In some embodiments, the antibody or antigen-binding fragment described herein comprises natalizumab.

In some embodiments, the antibody or antigen binding fragment described herein comprises a monoclonal antibody. Examples of monoclonal antibodies include, but are not limited to 3F8, Abagovomab, Abatacept, Abciximab, AC7885, Adalimumab, Adecatumumab, Afelimomab, Aflibercept, Afutuzumab, Alacizumab, Alemtuzumab, Altumomab, Anatumomab, Anrukinzumab, Apolizumab, Arcitumomab, Aselizumab, Atlizumab, Atorolimumab, Bapineuzumab, Basiliximab, Bavituximab, Bectumomab, Belatacept, Belimumab, Bertilimumab, Besilesomab, Bevacizumab, Biciromab, Bivatuzumab, Blinatumomab, Canakinumab, Cantuzumab, Capromab, Catumaxomab, Cedelizumab, Certolizumab, Cetuximab Erbitux, Citatuzumab, Cixutumumab, Clenoliximab, CNTO 1275 (=ustekinumab), CNTO 148 (=golimumab), Conatumumab, Dacetuzumab, Dacliximab (=daclizumab), Daclizumab, Denosumab, Detumomab, Dorlimomab, Dorlixizumab, Ecromeximab, Eculizumab, Edobacomab, Edrecolomab, Efalizumab, Efungumab, Elsilimomab, Enlimomab, Epitumomab, Epratuzumab, Erlizumab, Ertumaxomab, Etanercept, Etaracizumab, Exbivirumab, Fanolesomab, Faralimomab, Felvizumab, Figitumumab, Fontolizumab, Foravirumab, Galiximab, Gantenerumab, Gavilimomab, Gemtuzumab, Golimumab, Gomiliximab, Ibalizumab, Ibritumomab, Igovomab, Imciromab, Infliximab Remicade, Inolimomab, Inotuzumab ozogamicin, Ipilimumab, Iratumumab, Keliximab, Labetuzumab, Lebrilizumab, Lemalesomab, Lerdelimumab, Lexatumumab, Libivirurnab, Lintuzumab, Lucatumumab, Lumiliximab, Mapatumumab, Maslimomab, Matuzumab, Mepolizumab, Metelimumab, Milatuzumab, Minretumomab, Mitumomab, Morolimumab, Motavizumab, Muromonab, MYO-029, Nacolomab, Naptumomab, Natalizumab, Nebacumab, Necitumumab, Nerelimomab, Nimotuzumab, Nofetumomab, Ocrelizumab, Odulimomab, Ofatumumab, Omalizumab, Oportuzumab, Oregovomab, Otelixizumab, Pagibaximab, Palivizumab, Panitumumab, Panobacumab, Pascolizumab, Pemtumomab, Pertuzumab, Pexelizumab, Pintumomab, Priliximab, Pritumumab, PRO 140, Rafivirumab, Ramucirumab, Ranibizumab, Raxibacumab, Regavirumab, Reslizumab, Rilonacept, Rituximab, Robatumumab, Rovelizumab, Rozrolimupab, Ruplizumab, Satumomab, Sevirumab, Sibrotuzumab, Siltuximab, Siplizumab, Solanezumab, Sonepcizumab, Sontuzumab, Stamulurnab, Sulesomab, Tacatuzumab, Tadocizumab, Talizumab, Tanezumab, Tapliturnomab, Tefibazumab, Telimomab, Tenatumomab, Teneliximab, Teplizumab, TGN1412, Ticilimumab (=tremelimumab), Tigatuzumab, TNX-355 (=ibalizumab), TNX-650, TNX-901 (=talizumab), Tocilizumab, Toralizumab, Tositumomab, Trastuzumab, Tremelimumab, Tucotuzumab, Tuvirumab, Urtoxazumab, Ustekinumab, Vapaliximab, Vedolizumab, Veltuzumab, Vepalimomab, Visilizumab, Volociximab, Votumumab, Zalutumumab, Zanolimumab, Ziralimumab, and Zolimomab.

In some embodiments, the antibody is adalimumab, infliximab, tocilizumab, vedolizumab, eculizumab, alemtuzumab, natalizumab, atezolizumab, bevacizumab, cetuximab, daratumumab, ipilimumab, nivolumab, obinutuzumab, pembrolizumab, pertuzumab, ramucirumab, rituximab, trastuzumab, golimumab, ustekinumab, denosumab, certolizumab pegol, secukinumab, or blinatumomab.

In another aspect, provided herein are methods for treating a disease or condition in a subject. In some embodiments, the methods comprises (a) determining a level of an analyte in a serum of the patient, comprising: (i) perturbing a soft tissue in a mouth or other cavity of the patient; (ii) collecting a sample of fluid from the mouth or other cavity of the patient; and (iii) measuring the level of the analyte in the sample of fluid from the mouth or other cavity of the patient. In some embodiments, measuring the level of the analyte in the sample of fluid from the mouth or other cavity of the patient determines the level of the analyte in the serum of the patient. In some embodiments, the method comprises administering an anti-drug antibody or antigen-binding fragment thereof to the subject.

In some embodiments, the anti-drug antibody or antigen-binding fragment thereof binds to a therapeutic antibody. In some embodiments, the anti-drug antibody or antigen-binding fragment thereof comprises an anti-idiotypic antibody to the therapeutic antibody. In some embodiments, the anti-idiotypic antibody comprises an antigen blocking anti-idiotype antibody, a non-blocking anti-idiotype antibody, or a complex specific anti-idiotype antibody.

In some embodiments, the disease or condition comprises virus infection, bacterial infection, autoimmune disease, corona virus, multiple sclerosis, polyomavirus, JC virus, progressive multifocal leukoencephalopathy, Guillain-Barré syndrome, Myasthenia Gravis, flu, monkey pox, AIDS or a combination thereof.

### Certain Definitions

Unless otherwise defined, all technical terms used herein have the same meaning as commonly understood by one of ordinary skill in the art. As used in this specification and the appended claims, the singular forms “a,” “an,” and “the” include plural references unless the context clearly dictates otherwise. Any reference to “or” herein is intended to encompass “and/or” unless otherwise stated. As used herein, the term “about” a number refers to that number plus or minus 10% of that number.

The term “about” or “approximately” can mean within an acceptable error range for the particular value as determined by one of ordinary skill in the art, which will depend in part on how the value is measured or determined, i.e., the limitations of the measurement system. For example. “about” can mean within 1 or more than 1 standard deviation, per the practice in the art. Alternatively, “about” can mean a range of up to 20%, up to 15%, up to 10%, up to 5%, or up to 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures. To facilitate an understanding of the present disclosure, a number of terms and phrases are defined below.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

As used herein, a “mimetope” is a determinant which is recognized by the same binding molecule, such as an antibody, as a particular “epitope” but which has a different composition from the “epitope.” For example, a binding molecule can be an antibody which recognizes (i.e., binds to) an epitope comprising a linear sequence of amino acids. A “mimetope” of this epitope comprises a different linear sequence of amino acids but which is still recognized by the same antibody. In some embodiments, the mimetope is a Veritope™.

As used herein, “polypeptide” and “peptide” are used broadly to refer to macromolecules comprising linear polymers of natural or synthetic amino acids. Polypeptides may be derived naturally or synthetically by standard methods known in the art. While the term “polypeptide” and “peptide” are synonymous, the term “polypeptide” generally refers to molecules of greater than 40 amino acids, while the term “peptide” generally refers to molecules of 2 to 40 amino acids. In some embodiments, the peptide is a mimetope.

The term “antibody” as used herein refers to immunoglobulin molecules and immunologically active portions of immunoglobulin molecules, i.e., molecules that contain an antigen binding site that immunospecifically binds an antigen. The term also refers to antibodies comprised of two immunoglobulin heavy chains and two immunoglobulin light chains as well as a variety of forms including full length antibodies and portions thereof; including, for example, an immunoglobulin molecule, a monovalent antibody, an intact antibody, a bivalent antibody, a scrambled antibody, total antibody, a polyclonal antibody, a monoclonal antibody (mAb), a recombinant antibody, a chimeric antibody, a humanized antibody, a camelid antibody, a nanobody, a VHH antibody, a CDR-grafted antibody, F(ab)2, Fv, scFv, IgGΔCH2, F(ab′)2, scFv2CH3, F(ab), VL, VH, scFv4, scFv3, scFv2, dsFv, Fv, scFv-Fc, (scFv)2, a disulfide linked Fv, a single domain antibody (dAb), a diabody, a multispecific antibody, a dual specific antibody, an anti-idiotypic antibody, a bispecific antibody, any isotype (including, without limitation IgA, IgD, IgE, IgG, or IgM), a modified antibody, and a synthetic antibody (including, without limitation non-depleting IgG antibodies, T-bodies, or other Fc or Fab variants of antibodies). As used herein, the term “bivalent” is referred to as “intact” and the term “monovalent” is referred to as “scrambled.”

The term “antigen-binding domain” is used to refer to one or more antibody variable domain(s) (e.g., formed from amino acids from a single polypeptide or formed from amino acids from two or more polypeptides (e.g., the same or different polypeptides) that is capable of specifically binding to one or more different antigen(s). In some examples, an antigen-binding domain can bind to an antigen or epitope with specificity and affinity similar to that of naturally-occurring antibodies. In some embodiments, the antigen-binding domain can be an antibody or a fragment thereof. In some embodiments, an antigen-binding domain can include an alternative scaffold. Non-limiting examples of antigen-binding domains are described herein. Additional examples of antigen-binding domains are known in the art.

The term “antigen-binding fragments” refers to a portion of a full-length antibody or a polypeptide that includes a portion of a full-length antibody, that retains antigen-binding activity via its variable region or regions. Examples of antigen-binding fragments include Fab, Fab′, F(ab′)2, and Fv fragments; diabodies; linear antibodies; minibodies (Olafsen et al., Protein Eng. Design & Sel. 17(4): 315-323, 2004), fragments produced by a Fab expression library, single-chain antibody molecules; and multispecific antibodies formed from antigen-binding fragments.

The term “complementarity determining region” or “CDR” refers to one of the three hypervariable regions (or HVRs) that are known to confer (at least in part) antigen-binding specificity in each antibody light chain and each antibody heavy. The three CDRs in the antibody heavy chain and the antibody light chain interrupt four framework regions in the heavy chain variable domain and the light chain variable domain. The CDRs of each chain are typically referred to as CDR1, CDR2, and CDR3, numbered sequentially starting from the N-terminus, and are also typically identified by the chain in which the particular CDR is located.

The “framework regions” or “FRs” of different light immunoglobulin chains and different heavy immunoglobulin chains are relatively conserved within different antibodies produced by a mammal. The framework regions of light and heavy immunoglobulin chains serve to position and align the CDRs in three-dimensional space. Framework sequences can be obtained from public DNA databases or published references that include germline antibody gene sequences. For example, germline DNA sequences for human heavy and light chain variable region genes can be found in the “VBASE2” germline variable gene sequence database for human and mouse sequences.

“Monospecific” antigen-binding protein refers to the ability of the antigen-binding protein, such as an antibody, to bind only one epitope. “Bispecific” antigen-binding protein refers to the ability of the antigen-binding protein to bind two different epitopes. “Multispecific” antigen-binding protein refers to the ability of the antigen-binding protein to bind more than one epitope. In certain embodiments, a multispecific antigen-binding protein, such as a multispecific antibody, encompasses a bispecific antigen-binding protein or a bispecific antibody. For bispecific and multispecific antigen-binding proteins provided herein, the epitopes can be on the same antigen, or each epitope can be on a different antigen. Therefore, in certain embodiments, a multispecific antigen-binding protein provided herein, such as a bispecific antibody, binds to two different antigens. In certain embodiments, the multispecific antigen-binding protein, such as a bispecific antibody, binds to different epitopes on one antigen. In certain embodiments, a multispecific antigen-binding protein provided herein binds to each epitope with a dissociation constant (Kd) of about <1 M, about <100 nM, about <10 nM, about <1 nM, about <0.1 nM, about <0.01 nM, or about <0.001 nM (e.g., about 10⁻⁸ M or less, e.g., from about 10⁻⁸ M to about 10⁻¹³M, e.g., from about 10⁻⁹ M to about 10⁻¹⁰ M).

The term “affinity” refers to the strength of the sum of all non-covalent interactions between an antigen-binding site and its antigen. Unless otherwise indicated, “affinity” refers to intrinsic binding affinity, which reflects a 1:1 interaction between an antigen-binding domain and an antigen. Affinity can be measured, e.g., using surface plasmon resonance (SPR) technology (e.g., BIACORE®) or biolayer interferometry (e.g., FORTEBIO®). Additional methods for determining the affinity of an antigen-binding domain and its antigen are known in the art.

The term “single-chain polypeptide” means a polypeptide comprising a single polypeptide chain.

The term “multi-chain polypeptide” means a complex of two or more (e.g., 2, 3, 4, 5, 6, 7, or 8) polypeptide chains (e.g., the same or different polypeptide chains) that covalently and/or non-covalently associate with each other. For example, two or more polypeptide chains of a multi-chain polypeptide can associate through the use of two domains that associate with each other (e.g., two Fc domains or IL-15 and the sushi domain of IL-15 receptor alpha).

A “pharmaceutically acceptable excipient, carrier or diluent” refers to an excipient, carrier or diluent that can be administered to a subject, together with an agent, and which does not destroy the pharmacological activity thereof and is nontoxic when administered in doses sufficient to deliver a therapeutic amount of the agent. A “pharmaceutically acceptable salt” suitable for the disclosure may be an acid or base salt that is generally considered in the art to be suitable for use in contact with the tissues of human beings or animals without excessive toxicity, irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzene sulfonic, ethane disulfonic, 2-hydroxyethyl sulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH2) n-COOH where n is 0-4, and the like. Similarly, pharmaceutically acceptable cations include, but are not limited to sodium, potassium, calcium, aluminum, lithium and ammonium. Those of ordinary skill in the art will recognize from this disclosure and the knowledge in the art that further pharmaceutically acceptable salts include those listed by Remington's Pharmaceutical Sciences, 17th ed., Mack Publishing Company, Easton, PA, p. 1418 (1985). In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in an appropriate solvent.

The term “administer” refers to a method of delivering compositions to the desired site of biological action. These methods include, but are not limited to, topical delivery, parenteral delivery, intravenous delivery, intradermal delivery, subcutaneous delivery, intramuscular delivery, colonic delivery, rectal delivery, or intraperitoneal delivery.

The term “subject” refers to an animal which is the object of treatment, observation, or experiment. By way of example only, a subject includes, but is not limited to, a mammal, including, but not limited to, a human or a non-human mammal, such as a non-human primate, bovine, equine, canine, ovine, or feline.

The term “optional” or “optionally” denotes that a subsequently described event or circumstance can but need not occur, and that the description includes instances where the event or circumstance occurs and instances in which it does not.

As used herein, the terms “sample” and “biological sample” refer to any sample suitable for the methods provided by the present invention. In one embodiment, the biological sample of the present invention is a physiological fluid, for example, whole blood or fraction thereof (e.g., serum or plasma), urine, spinal fluid, saliva, nasal, vaginal fluid, and stool. In some embodiments, the sample is saliva.

As used herein, the term “solid support” refers to any solid phase material upon which a polypeptide, such as a mimetope, is synthesized or attached, such as conjugation via covalent bond. Solid support encompasses terms such as “resin,” “solid phase,” and “support.”

The section headings used herein are for organizational purposes only and are not to be construed as limiting the subject matter described.

### EXAMPLE

The application may be better understood by reference to the following non-limiting examples, which are provided as exemplary embodiments of the application. The following examples are presented in order to more fully illustrate embodiments and should in no way be construed, however, as limiting the broad scope of the application.

### Example 1. Measurement of Covid Antibodies in Saliva and Nasal Samples

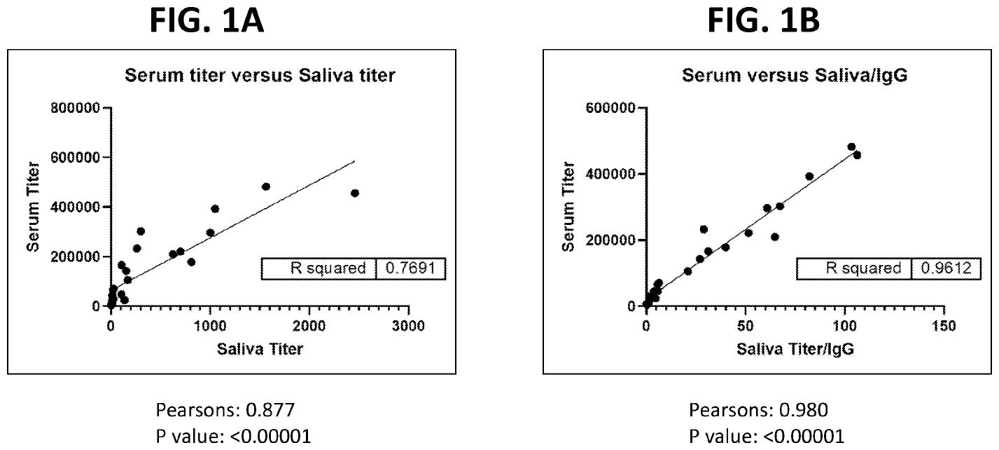

FIGS. 1A-1B are graphs showing that normalization of the saliva Spike S1 titer to total IgG present in saliva sample correlated to serum Spike S1 titer. Paired serum and saliva samples from donors were serial diluted on Spike S1 coated ELISA plates and the covid antibody titer obtained. The raw titer of covid antibodies in serum and saliva are compared in FIG. 1A. The saliva titer is normalized to total IgG content and is expressed as saliva titer per IgG (ug/ml) in FIG. 1B. This data shows a much better correlation to serum titers and demonstrates the importance of using IgG to correct for variations in the serum antibody concentration of saliva.

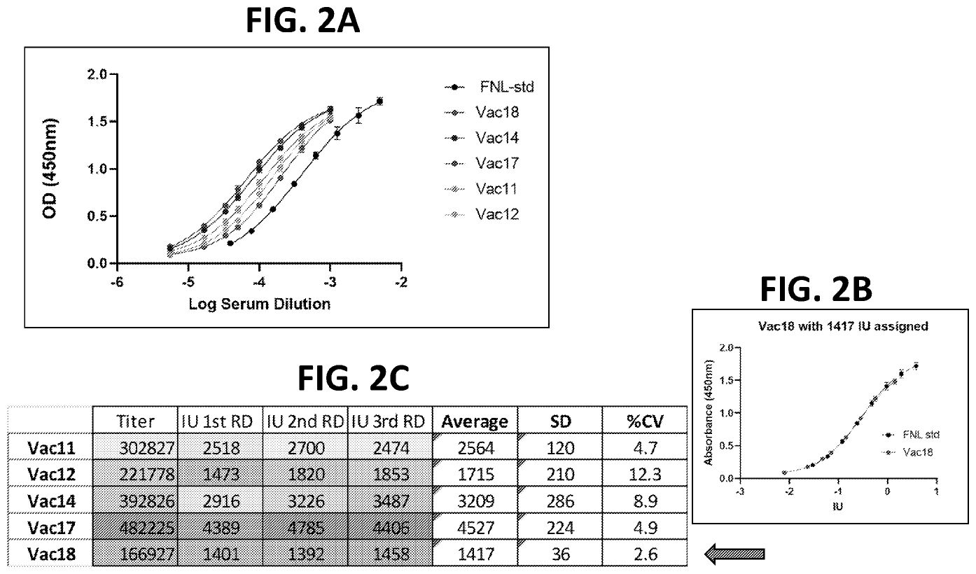

FIGS. 2A-2C provide graphs showing the obtained standard in WHO International Units (IU) using serum donations. The WHO standard is 1000 IU (Binding Antibody Units/ml) and the US (FNL) standard is 764 IU (BAU/ml). Five serum donors were analyzed with FNL-std in triplicate and on three separate days. The IU assigned was the mean obtained. The WHO international standard for COVID-19 antibodies was obtained and calibrated some of the donor serum samples to this standard for subsequent use as standards.

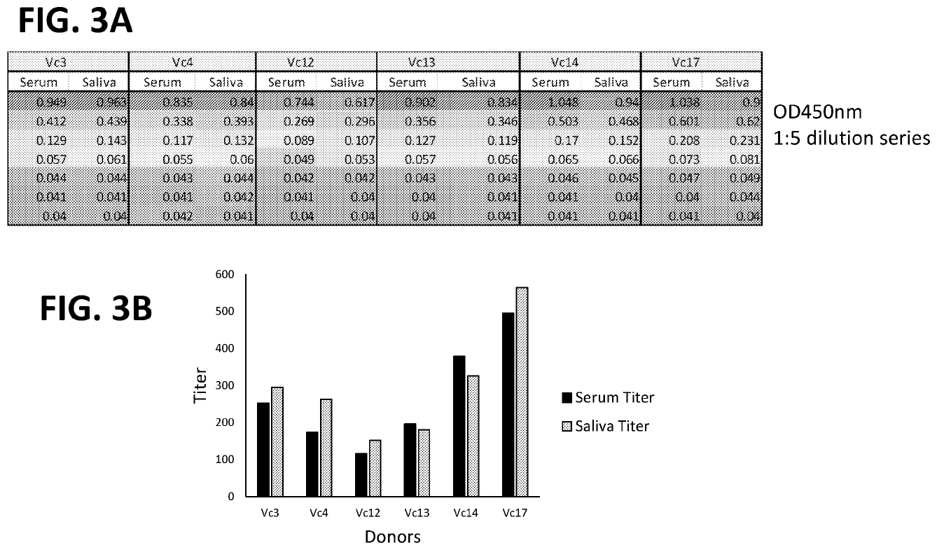

FIGS. 3A-3B are graphs showing similar levels of covid antibodies in saliva and serum when normalized to IgG levels. Serum was diluted to the same IgG concentration as a paired saliva sample in FIG. 3A. The total IgG concentration of serum and saliva was determined first. Then the serum sample was diluted down so that it had the same total IgG concentration as that obtained in the paired saliva sample. The samples were then diluted on the ELISA plate as normal. Raw OD readings are shown in FIG. 3A while the titers obtained for each sample are shown in FIG. 3B. When the samples have equivalent total IgG, covid antibody titers obtained for both matrices' are similar. This indicates the importance of normalizing to IgG levels.

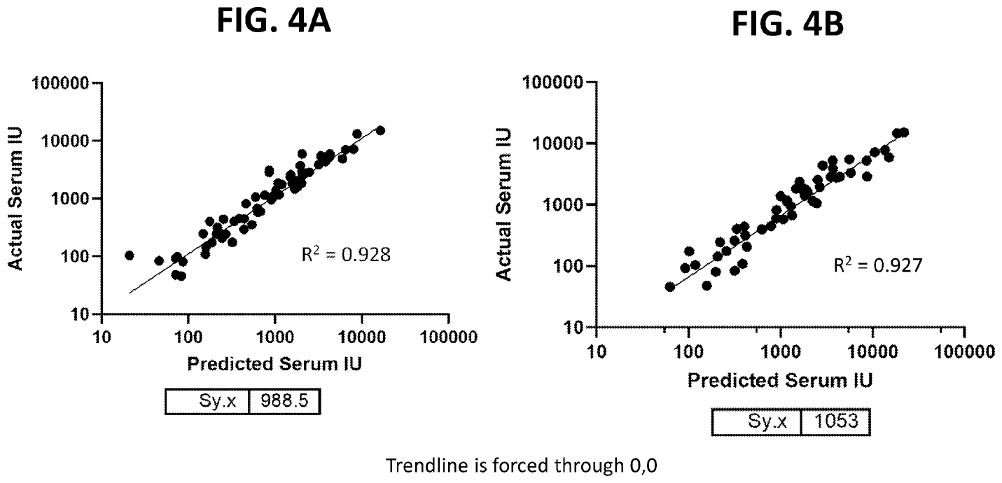

FIGS. 4A-4B are graphs showing the correlation between actual serum Spike S1 IU and that predicted from saliva (FIG. 4A) and nasal fluid (FIG. 4B). Serum IU was calculated from that measured in saliva samples by performing assays to obtain all matrix donations expressed in IU; then normalizing saliva and nasal samples to the total amount of IgG present in the sample (IU/IgG). An average total Serum IgG concentration was determined by calculating the mean IgG of all the serum donated, and this mean serum IgG concentration was used to predict what the serum Spike IU is from saliva or nasal samples. As an example, if the saliva IU is 50, and total IgG concentration is 5 ug/ml, then saliva IU/IgG=10, and if total mean of serum IgG concentration is 10 mg/ml, then predicted serum IU=10,000 IU. The serum covid-19 antibody level was predicted from saliva (FIG. 4A, x axis) and nasal fluid (FIG. 4B, x axis) and plotted against the actual measured serum level (y axis). Both matrix types show good predictive ability for serum antibody levels. Trendline is forced through 0,0.

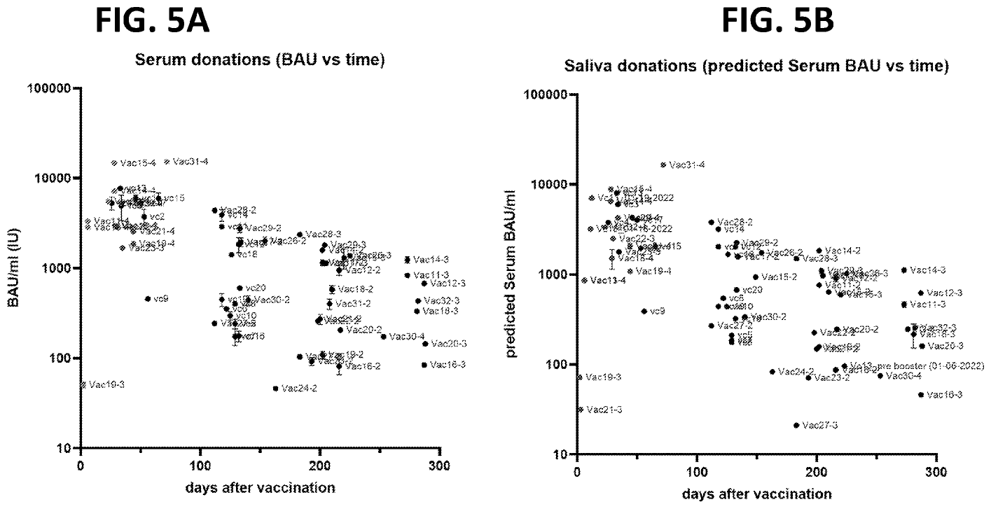

FIGS. 5A-5B are graphs illustrating serum (FIG. 5A) and saliva (FIG. 5B) predicted serum levels. Donor antibody levels (y axis) were tracked over time (x axis, days after vaccination). There is a decrease in antibody serum level the longer the time period from the vaccination date (FIG. 5A), and this decrease can also be seen using saliva as the matrix to predict serum antibody levels (FIG. 5B). Red data points indicate patients who received a vaccination booster shot.

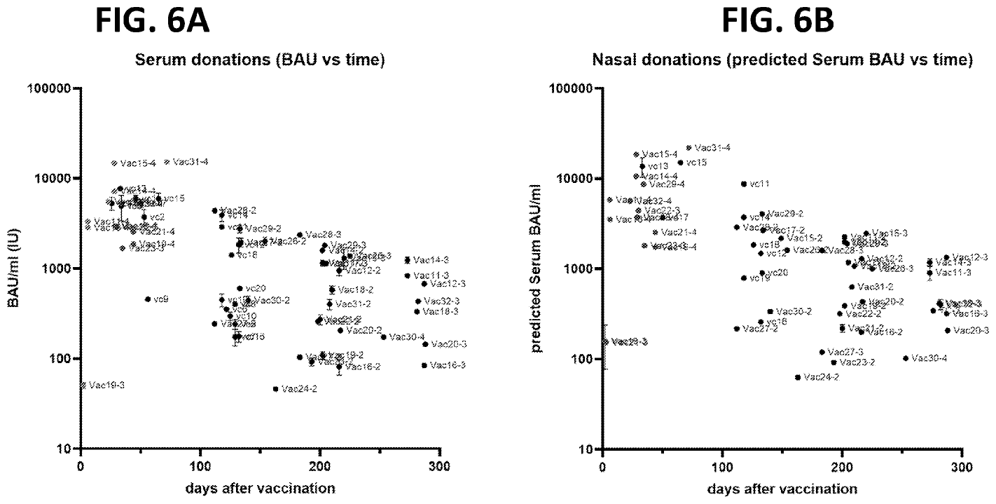

FIGS. 6A-6B are graphs illustrating serum (FIG. 6A) and nasal fluid (FIG. 6B) predicted serum levels. Donor antibody levels (y axis) were tracked over time (x axis, days after vaccination). There is a decrease in antibody serum level the longer the time period from the vaccination date (FIG. 6A), and this decrease can also be seen using nasal as the matrix to predict serum antibody levels (FIG. 6B). Red data points indicate patients who received a vaccination booster shot.

### Example 2. Collection of Samples

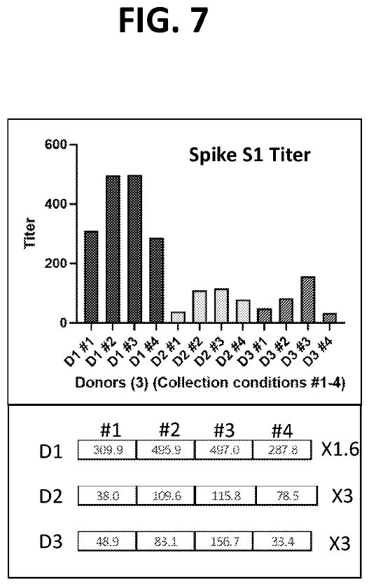

FIG. 7 depicts that the mechanism of saliva collection can influence the amount of IgG obtained. A test using three donors shows that the sample collection can be improved by more perturbation of the gums than the current collection process using an OraSure device (#1—regular: rub each gum 5× then place collection device between gum and cheek for 2 minutes). Both a more extended rubbing with a swab (#2—rub gums with swab for entire 2 minutes) and also a pre-collection step of brushing/flossing teeth (#3—brush teeth and floss before a regular collection as in #1) resulted in an increased concentration of covid antibodies detected. #4 is similar to #1 but half of the buffer in the collection tube was removed (800 ul down to 400 ul). In the saliva samples, there is a decreased antibody concentration compared to that seen in serum (˜100 fold) that led to reduced sensitivity—a collection device that allows for an increase in antibody concentration in the sample would help overcome this issue. Antibody is passively secreted in gingival crevicular fluid at the tooth/gum line so focused collection at that point rather than obtaining general saliva would be beneficial. The experiments disclosed herein indicate that rubbing gum/teeth line can help improve signal.

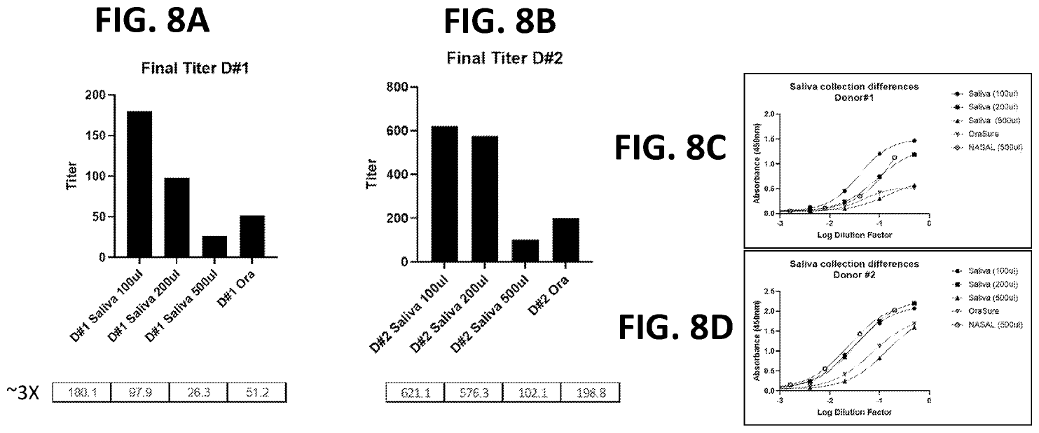

FIGS. 8A-8D depict that reducing the volume of buffer can cause an increase in antibody levels detected. This procedure involved a comparison of OraSure collection (800 ul buffer in collection tube) to using an iClean swab (iClean sampling swab with breakable handle/sterile) that was rubbed on the gums before being placed in a tube with various volumes of PBS (100 μl, 200 ul, or 500 ul). Reducing the volume of buffer caused an increase in antibody levels detected. This demonstrates that a collection device that can rub the gums and directly place the sample onto the lateral flow test strip can maximize sensitivity.

### Example 3. Lateral Flow Assay Test

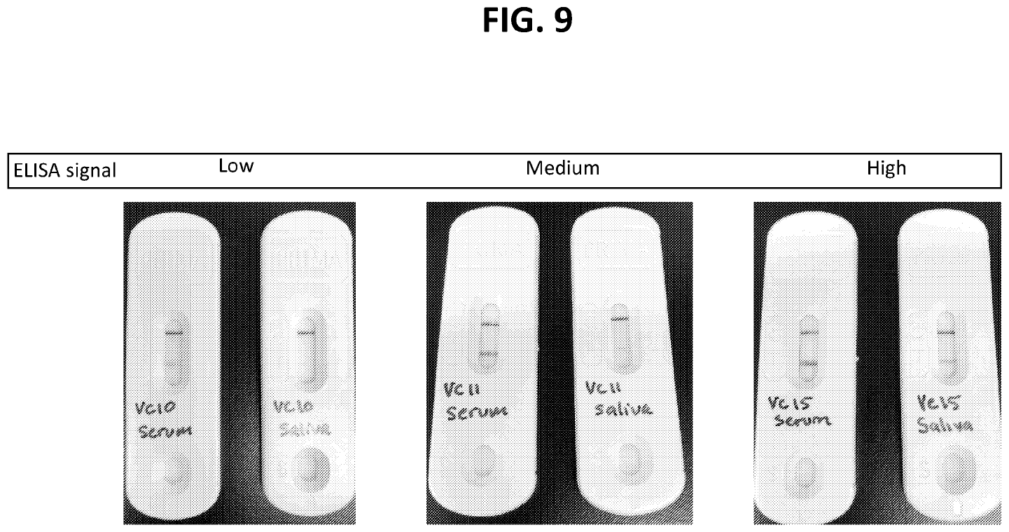

FIG. 9 depicts a comparison between serum vs saliva for a PRIMACOVID Covid-19 neutralizing antibody rapid test. A lateral flow serology test developed for detection of covid antibodies in serum was used to test compare the ability of testing for antibodies in saliva. Serum and paired saliva samples from individual donors that were shown by ELISA to have low, medium of high antibody levels were added to the devices and compared. Although the signal from saliva samples were lower than serum, the data for saliva samples showed the same low, medium, high pattern as seen in serum samples. These devices were not made to run saliva samples and tests geared towards this sample matrix will have improved signal intensities.

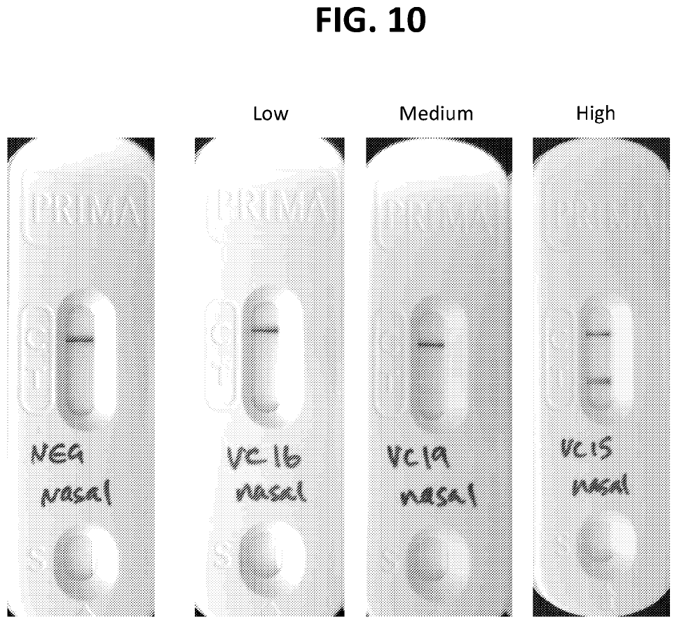

FIG. 10 depicts nasal fluid samples on a PRIMACOVID Covid-19 neutralizing antibody rapid test. Similar to the results depicted in FIG. 9. These data show that nasal fluid samples can also be run on these LFA strips to produce the expected pattern of signal intensities.

### Example 4. Measurement of TNF-Alpha in Saliva and Nasal Samples

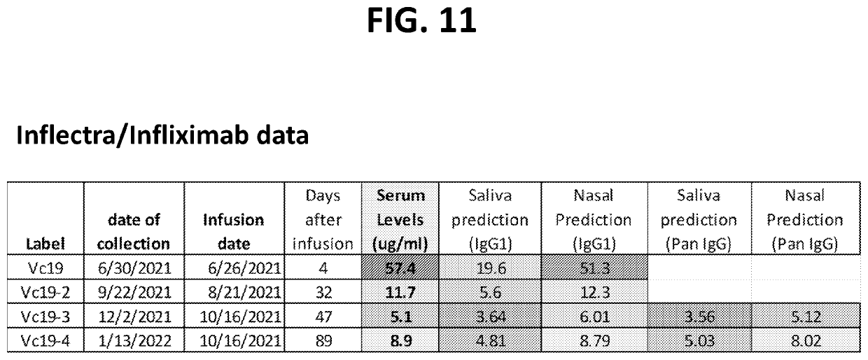

FIG. 11 illustrates that an infused therapeutic drug can be detected in saliva and nasal fluid. One of the donors in the covid antibody study was infused with Inflectra, a monoclonal antibody directed against TNF-alpha, every 2 months. The donor sample was tested to see whether the infused antibody could also be detected in saliva and nasal fluids. The ELISA plate was coated with TNF-alpha and Inflectra used to generate a standard curve to quantitate the level of drug in samples. Saliva and nasal samples were normalized to IgG and used to predict serum levels. FIG. 11A shows that Inflectra can be detected and quantitated in both saliva and nasal fluids in this donor, thus demonstrating that therapeutic drug monitoring using these non-invasive samples is possible.

### Example 5. ELISA Method

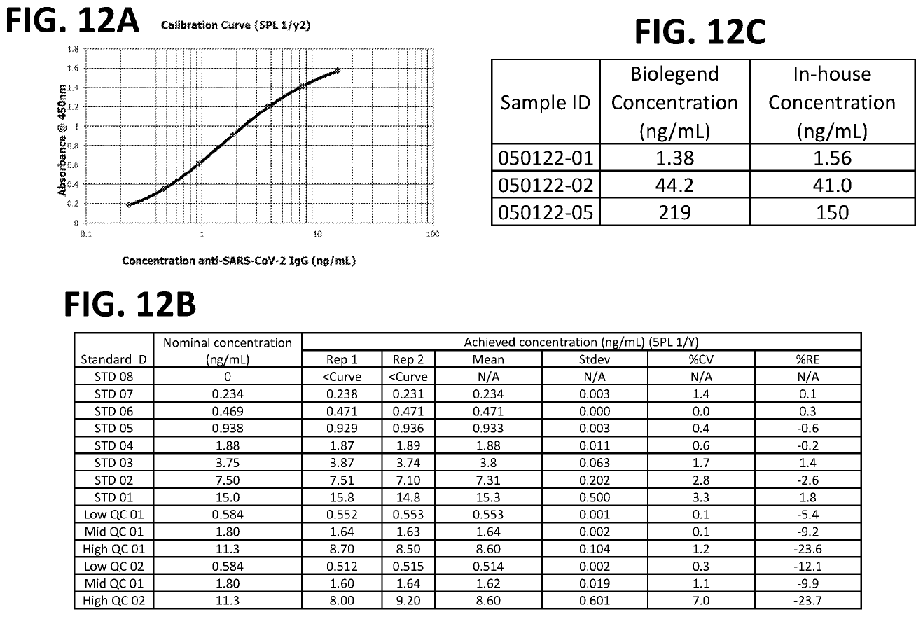

FIGS. 12A-12C demonstrate an exemplary ELISA method. The ELISA procedure used S1-Acro protein as capture and the calibration curve prepared using the positive control (Human anti-SARS-CoV-2 IgG supplied by The Native Antigen company, Cat: MAB12422-100). The results show acceptable performance for an unoptimized assay regarding precision (% CV) and accuracy (% RE) using similar reagents as for the lateral flow assay and included analysis of passive saliva samples for SARS-CoV-2 specific IgG. A review of saliva and serum samples (FIG. 12C) shows that back-calculated concentrations achieved with the exemplary ELISA method are comparable to off-the-shelf (Biolegend LEGEND MAX™ SARS-CoV-2 Spike S1 Human IgG ELISA kit). Serum sample analyzed diluted in assay buffer (PBSBT) between 1 in 50 to 1 in 25,600. Serum concentration back-calculated as 20,422 ng/ml (1417 BAU/mL). Serum concentrations (ng/mL) approximately 150-fold higher than the largest concentration achieved in saliva samples.

### Example 6. Lateral Flow Device

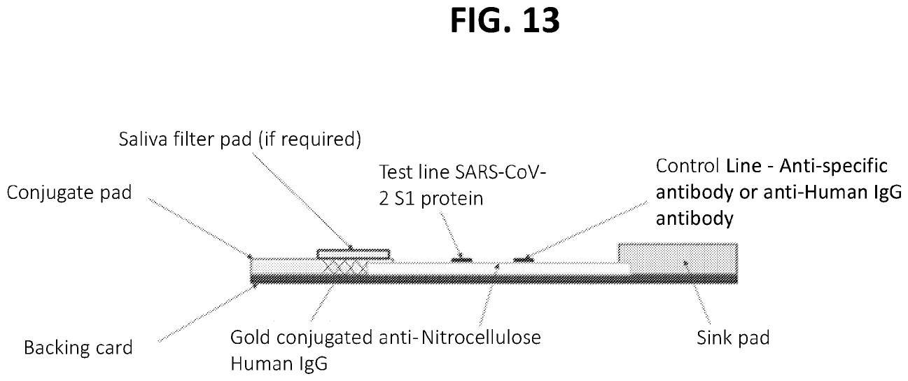

FIG. 13 depicts an exemplary lateral flow device, including S1 protein on nitrocellulose (NC) and detection with anti-human IgG coupled gold nanoparticles. This format allows for future inclusion of a secondary test line, measuring total IgG which could be a way of ensuring sufficient IgG in saliva sample monitoring the collection method and could also allow flexibility with changing test line protein considering SARS-CoV-2 variants. The current method of saliva collection is passive drool (which can yield low antibody levels and saliva dilution type and dilution requirement can impact wicking due to viscosity), whereas an exemplary approach disclosed herein includes a collection swab via rubbing of gums and elute with PBS (higher antibody levels from gingival crevicular fluid).

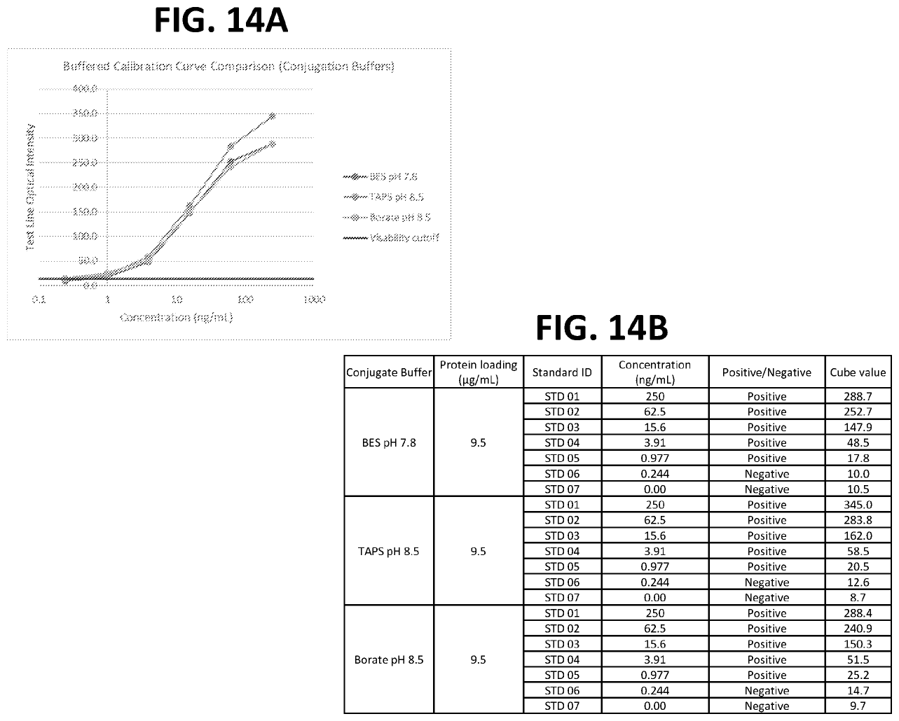

FIGS. 14A-B depicts the initial assessment of an exemplary lateral flow device format. These results were assessed in a generic assay buffer format and include a positive control, a human anti-SARS-CoV-2 IgG spiked into buffer, and the resulting test line intensity determined by Cube reader.

FIG. 15 depicts an exemplary collection device. As depicted, the toothbrush design is consumer friendly and stimulates small amounts of blood release into mouth. The modular design allows use as a platform for other tests, and connectivity via a mobile app and/or cloud data can be included.

### Example 7. Detection of Sars-CoV-2 Antibodies in Saliva and Nasal Samples

The Sars CoV-2 Spike S1 antibody levels were quantitated from paired serum, saliva and nasal specimens in a longitudinal study spanning a 10 month period post-vaccination. The cohort comprised of 31 vaccinated donors, 15 males and 16 females with an age range of 18-66 (median 42) and all participant specimens were collected with informed consent.

Serum was obtained using standard techniques (SST tubes, BD Vacutainer). For nasal mucosal collection, an iClean swab (Filtrous, #COV-12-0000) was placed ˜1 cm inside nostril and rotated for 10-15 seconds. Each nostril was sampled and then the swab placed into 500 ml of sterile PBS). Saliva collections were acquired employing a device (OraSure Technologies, #503-1111) that utilizes a swab to rub periodontal tissue, thus allowing for potential enrichment of serum antibodies at the expense of bulk saliva. The OraSure swab, was inserted into the mouth and rubbed against each gum/tooth line 5× (20× total) and then placed between gum and cheek for 2 minutes before processed as described by the manufacturer.

The ELISA assay developed to quantitate Covid-19 specific antibodies within the different matrices used a his-tagged SARS-CoV-2 S1 protein (AcroBiosystems # SIN-C52H3) bound to nickel coated plates (Thermo Scientific #15142) at a concentration of 1.5 ug/ml for 1 hour (all incubations described were performed at room temperature with agitation). Plates were washed 5 times with TBS/0.05% tween (TBST) using a Columbus Pro Tecan plate washer and blocked with addition of TBS/2.5% BSA for 1 hour. The extent of dilution of each matrix (into TBS/0.05% tween/2.5% BSA/1×Casein (PBSC-0100-01)) was determined empirically. After sample addition, plates were incubated for 1 hour, washed 5× with TBST, and then incubated with an anti-IgG antibody conjugated to HRP (Jackson #109-035-098) for 30 minutes. Following a TBST wash, Turbo TMB substrate (Thermofisher) was added, and reaction quenched at after 10 minutes with 1M H2SO4 (Thermofisher). The absorbance at 450 nM was read using the Multiskan FC plate reader (Thermofisher).

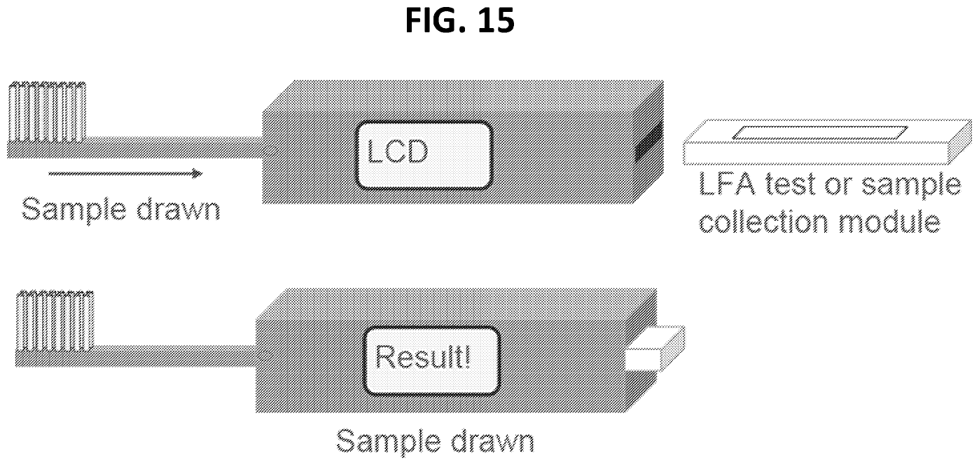

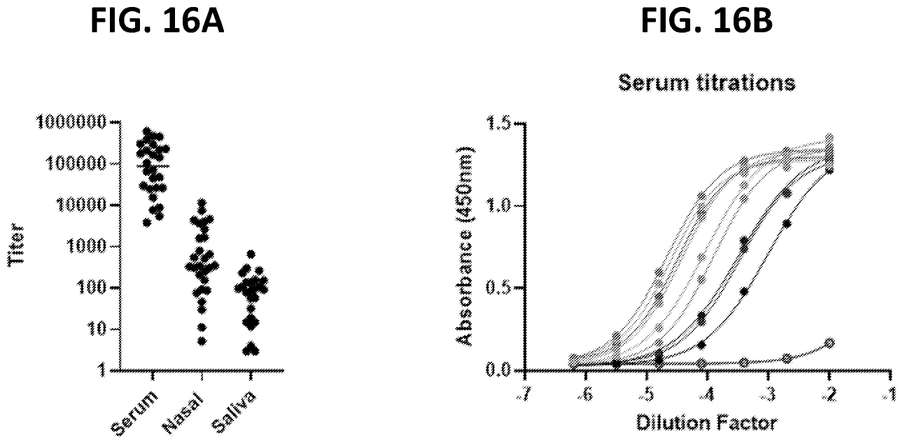

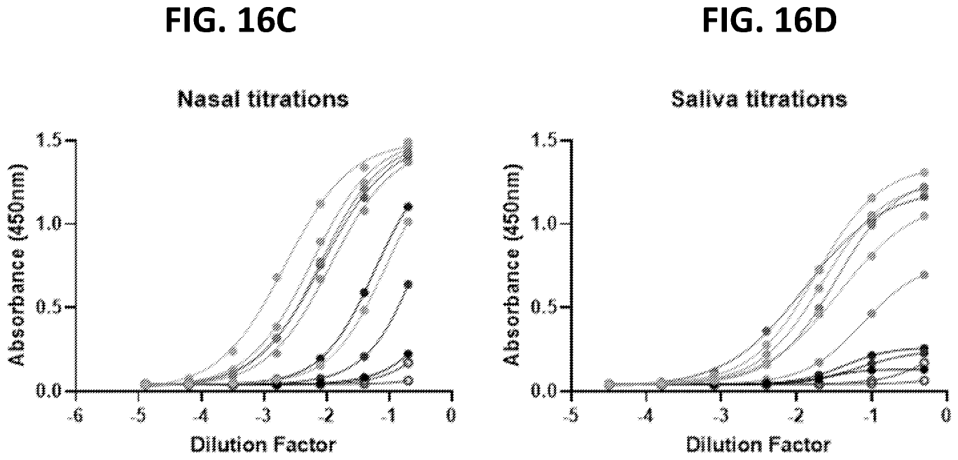

Although there were reduced levels in both nasal and saliva matrices compared to serum (FIG. 16A) quantifiable levels of antibodies directed against the SARS-CoV-2 Spike S1 protein were detected in most samples. Despite the differences in sensitivity between the 3 matrices, the identity of samples with high antibody levels, and those with low, remain the same although there is some switching of the rank order (FIGS. 16B-16D).

### Example 8. Normalization of Sars-Cov-2 Antibody Level to Total IgG Concentration in Saliva and Nasal Samples

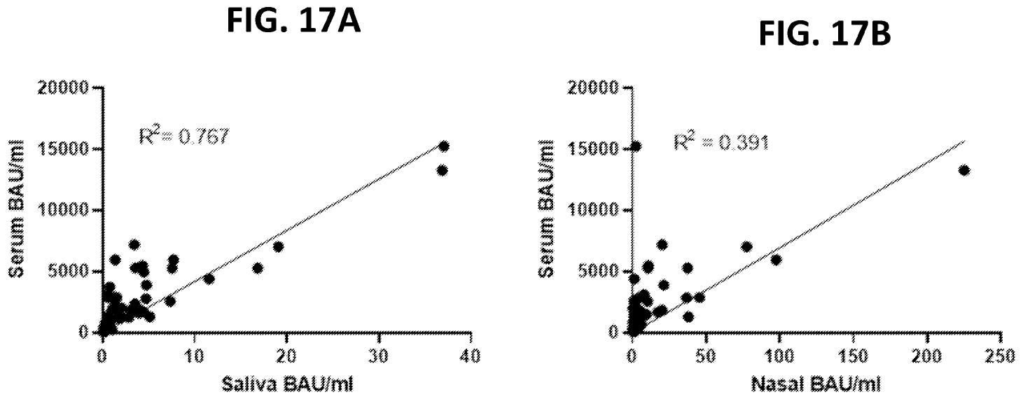

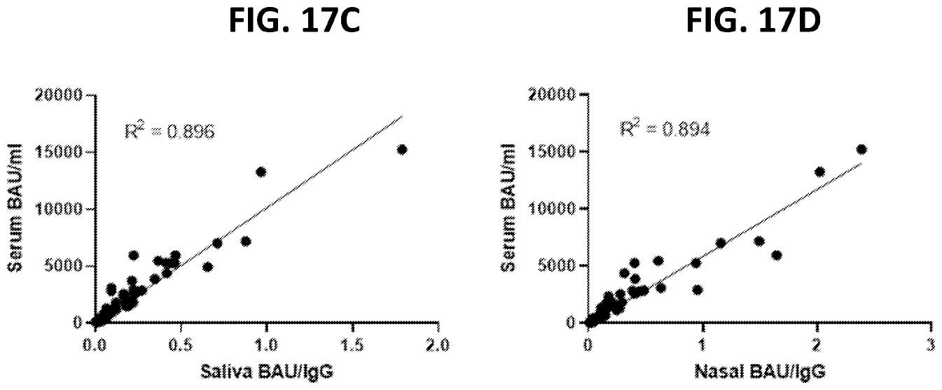

To standardize quantitation, the Sars CoV-2 Spike S1 antibody levels were quantified using standards calibrated to the WHO international standard for Spike IgG and are expressed as Binding Antibody Units (BAU). A comparison between the paired serum and saliva samples (FIG. 17A) and paired nasal and serum samples (FIG. 17B) showed some degree of correlation confirming the rough maintenance of the rank order seen in the raw data (as shown in FIG. 16).

Total IgG was measured in all samples (Mabtech #3850-1AD-6) and used to correct for the effects of these matrix specific factors. Details of the correction calculation are shown in Table 1 below.

TABLE 1
Example of Normalization of Specific Covid S1 Antibodies
in Saliva to Total IgG and Prediction of Serum Levels

| | | 3 | 4 | 5 | 6 |
|---|---|---|---|---|---|
| 1 | | Covid S1 | Average | Predicted | Measured |
| Covid S1 | 2 | specific IgG/ | donor total | serum Covid | serum Covid |
| specific IgG | Total IgG | Total IgG | serum IgG | S1 specific IgG | S1 specific IgG |
| (BAU/ml) | (ug/ml) | (BAU/ug) | (ug/ml) | (BAU/ml) | (BAU/ml) |
| 1.344 | 2.87 | 0.467 | 9181 | 4291 | 5952 |
| 0.333 | 4.5 | 0.074 | 9181 | 679 | 601 |
| 19.07 | 26.8 | 0.712 | 9181 | 6533 | 7223 |

The levels of Covid 1 specific antibodies were measured in the saliva samples using ELISA (column 1). The levels of total IgG were measured in the saliva samples (column 2). The specific Covid S1 antibody levels were divided by the total IgG found in the saliva samples to give the specific antibody measurement per ug of total IgG (column 3). To predict the serum levels from that detected in saliva, the average total IgG concentration obtained for all serum donations (column 4) was used to multiply the specific Covid S1/total IgG in saliva (column 3). The predicted serum (column 5) obtained from this calculation was compared to the value measured in the paired serum sample (column 6).

As shown in FIGS. 17C-17D, a much-improved correlation was observed when the saliva and nasal samples are normalized to the total IgG concentration present in the sample matrix before being compared to the pared serum sample.

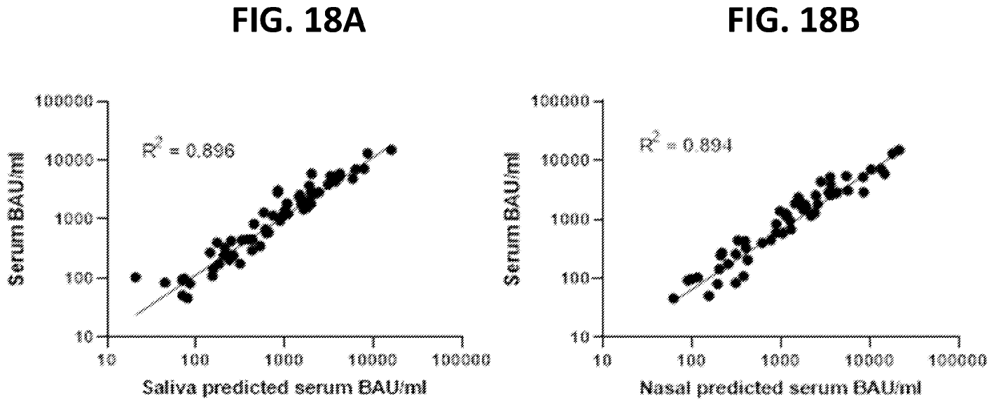

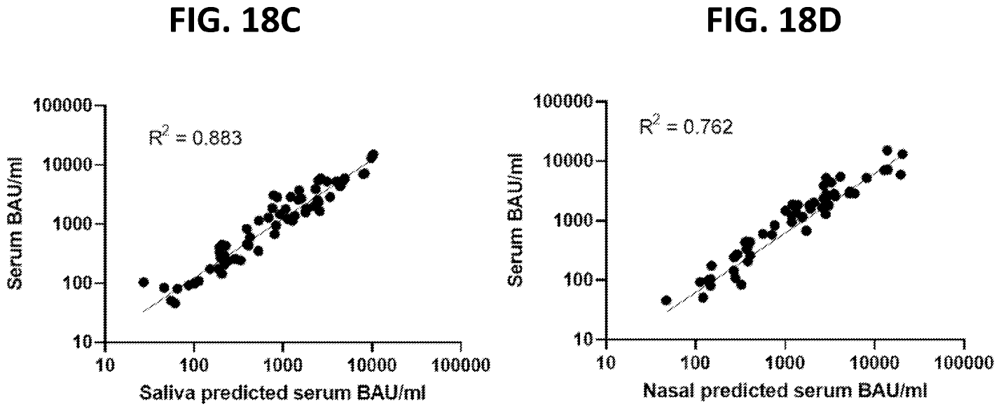

Since the total IgG normalized saliva and nasal samples showed good correlation to serum Sars-CoV-2 antibody levels (Pearson r=0.947 and 0.945 respectively), a correction of the samples analyte measurement to reflect a typical serum total IgG concentration should allow for a prediction of Sars CoV-2 antibody levels in the serum. The total IgG concentration in human serum can differ by ˜2.5-fold (ranging from 6-16 mg/ml) so both the serum IgG concentration of the appropriate paired donor serum sample, as well as the average IgG concentration of all donated serum, were used to predict the specific Covid IgG concentration in serum. Both the paired serum IgG concentration (FIGS. 18C-18D, saliva r=0.94, nasal r=0.873) and the average cohort serum IgG concentration (FIGS. 18A-18B, saliva r=0.947, nasal r 0.945) gave good concordance between predicted and measured serum Sars CoV-2 antibody levels.

### Example 9. Measurements of Sars-Cov-2 Antibodies Post Vaccination

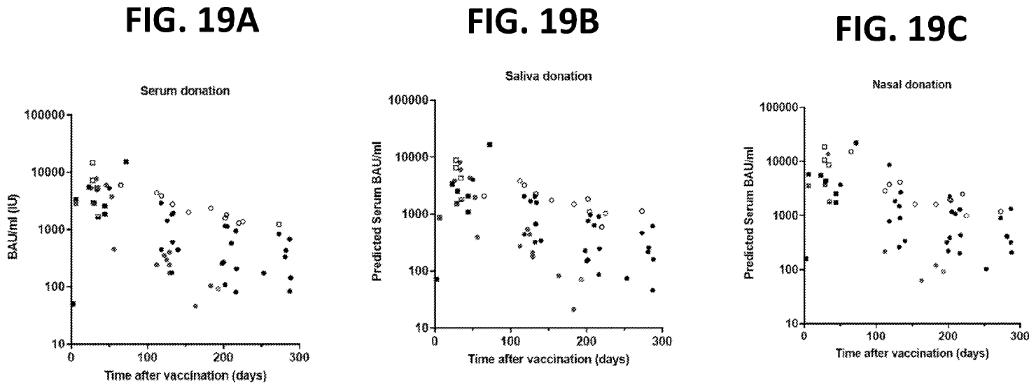

Sars-CoV-2 S1 antibody levels in donated serum (FIG. 19A), serum levels determined from saliva (FIG. 19B), serum levels determined from nasal samples (FIG. 19C) are plotted against time after the last vaccination.

As shown in FIG. 19A, Sars-CoV-2 antibody levels of vaccinated subjects declines overtime. A Sars-CoV-2 infection prior to vaccination generated the highest antibody levels and vaccination with Moderna resulted in less of a decline in antibody levels over time in comparison with vaccination with Pfizer. Most importantly, the predicted serum anti-Spike S1 antibody levels obtained from measurements using saliva and nasal samples behaved in a similar way to actual serum measurements (FIGS. 19B-19C).

### Example 10. Measurement of Natalizumab in Saliva and Nasal Samples

The data shows that the levels of natalizumab in saliva correlates well with those found in serum and can be used to predict serum levels. Patients diagnosed with multiple sclerosis that have been undergoing natalizumab therapy uninterrupted for the last 6 months were provided. Each consenting subject provides 1 tube of blood (serum) and a saliva sample at the following time points: visit 1—just prior to their scheduled infusion of natalizumab; visit 1—90 minutes post-infusion; visit 2—two weeks after their visit 1 infusion; visit 3—just prior to their subsequent natalizumab therapy (˜35 days for subjects on standard interval dosing or ˜45 days for subjects on extended interval dosing); and visit 3—90 minutes after natalizumab therapy. Both the blood and saliva samples were used in ELISA assays to measure levels of natalizumab, IgG4, total IgG, and antibodies against SARS-CoV-2.

Saliva samples were collected using a saliva collection device (for example, the OraSure Collection Device (OraSure Technologies, #503-1111). In the case of OraSure, the device consists of a swab and a collection tube containing a preservative buffer. The OraSure swab, was inserted into the mouth and rubbed against each gum/tooth line.

Natalizumab concentrations (total, monovalent, and bivalent), and total IgG4 were measured by enzyme-linked immunosorbent assays (ELISAs) developed by Abreos Biosciences. Total IgG was measured using a commercially available ELISA. The data obtained for natalizumab concentrations were normalized to the total IgG present in each sample to correct for differences in the amount of blood present in donors saliva.

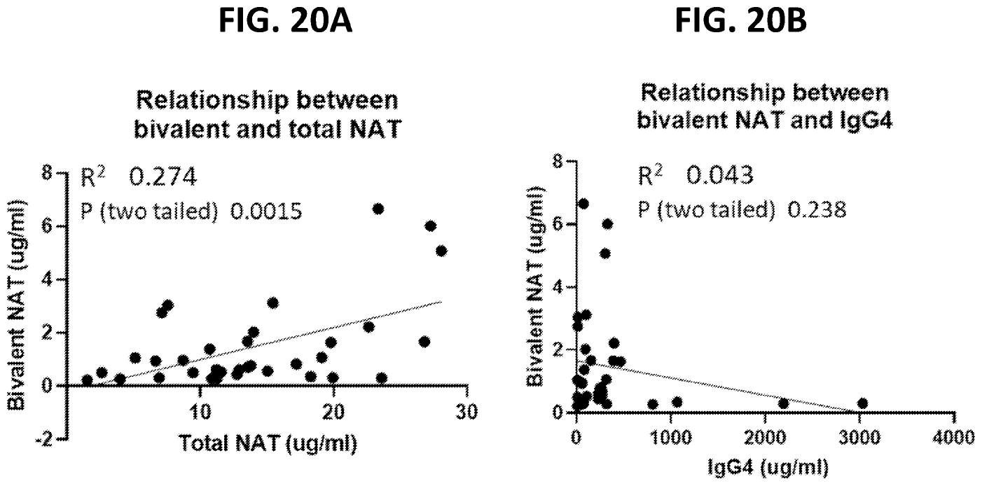

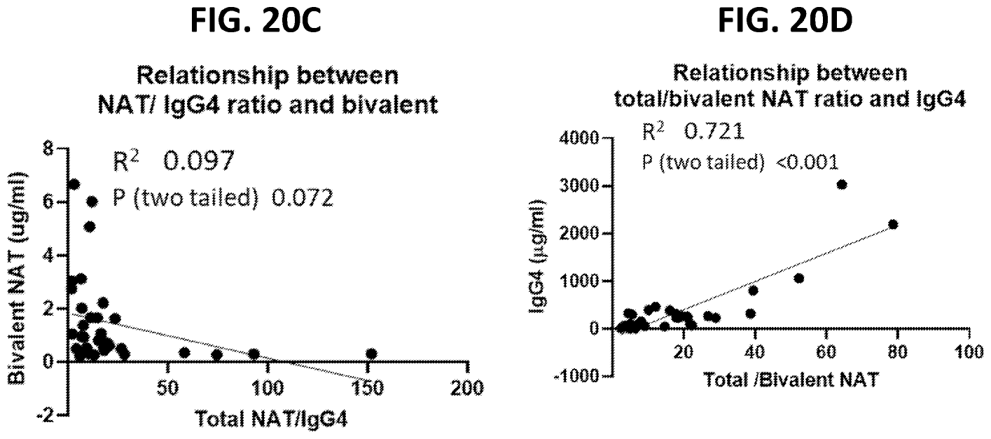

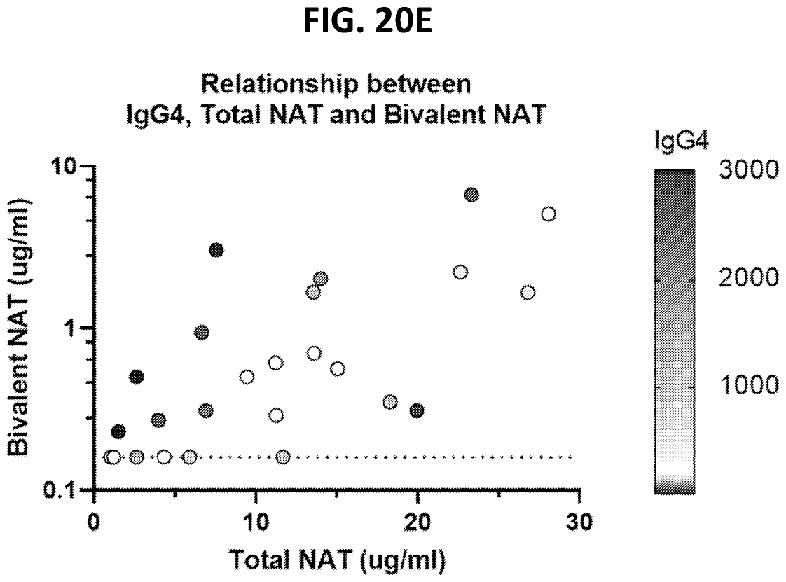

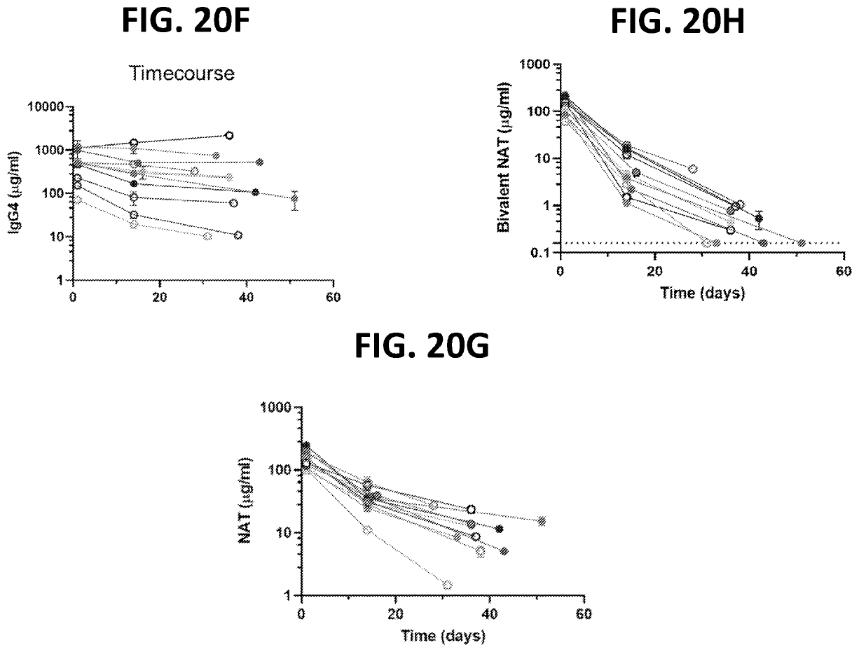

Although there is a significant correlation between bivalent NAT and total NAT at trough levels (FIG. 20A) it is quite weak (R²=0.3) indicating that other factors are affecting the amounts of bivalent present. When looking at the effect of IgG4 on bivalent levels no significant correlation was observed (FIG. 20B), however, there is a weak but significant correlation between the total NAT/IgG4 ratio and bivalent levels (FIG. 20C) and a strong correlation between the total/bivalent NAT ration and bivalent levels (FIG. 20D, R²=0.72). This indicates that while total NAT levels can influence the level of bivalent NAT, IgG4 levels in an individual can dictate how much of the total NAT is converted into bivalent. This relationship is shown more clearly in the multiple variable graph displayed in FIG. 2 E. Samples that have low IgG4 (blue) tend to have increased bivalent at a given total NAT concentration than samples with average IgG4 (white) samples, while those with high IgG4 (red) tend to have lower bivalent levels. This data set indicates the importance of monitoring bivalent levels, as there are subjects with relatively low total NAT trough levels (below 10 ug/ml) yet due to low IgG4 levels they contain unexpectedly high bivalent levels.

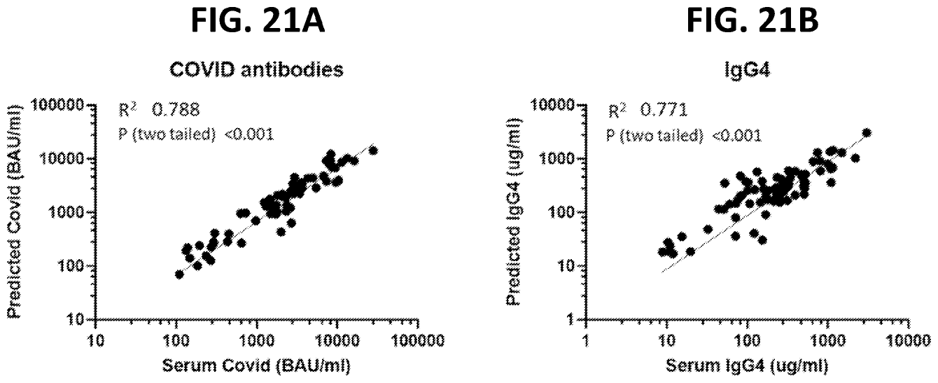

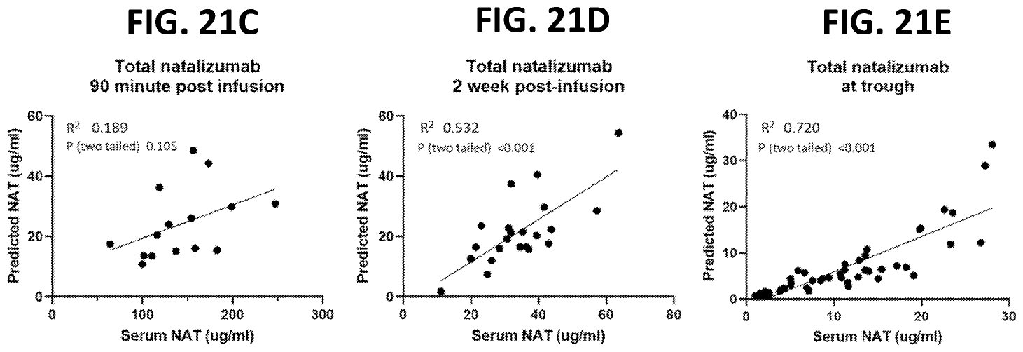

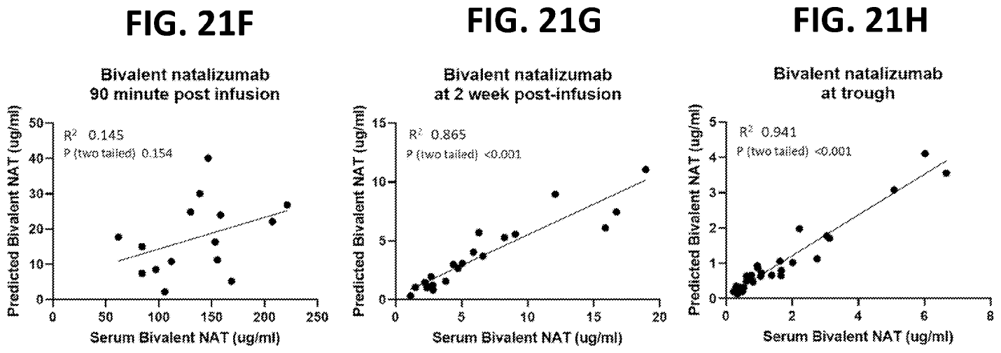

The ability to measure total and bivalent NAT in saliva and use that to determine serum levels will greatly facilitate the ability to monitor drug levels in an at-home-setting. FIG. 21A shows that, using an IgG calibrator method, there is a good correlation between the serum covid antibody levels predicted from measurements in saliva and those of direct serum measurements. As shown in FIG. 21B, the serum titer is normalized to total IgG content and is expressed as saliva titer per IgG (ug/ml). Interestingly it was found that the time of measurement after infusion is important as there is no correlation for either total or bivalent NAT 90-minutes post-infusion, with both saliva assays greatly underpredicting (FIG. 21C and FIG. 21F). This suggests that there is a time period required for equilibration between saliva and serum. The correlation appears to improve with increased time after infusion. Both the saliva total and bivalent NAT assays underpredict serum levels by ˜2-fold, which can be corrected for, but suggests that there may be some biological reason behind this. It is important to note that the ELISA assays have not been fine tuned to compensate for differences in serum and saliva matrices but rather the same assay format was used in each case. Despite this, good correlations were seen at trough (FIG. 21E and FIG. 21F), particularly for the bivalent assay (FIG. 21H).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of determining a level of an analyte in a serum of a patient in need thereof, comprising:
a. perturbing a soft tissue in a mouth or other cavity of the patient;
b. collecting a sample of fluid from the mouth or other cavity of the patient; and
c. measuring the level of the analyte in the sample of fluid from the mouth or other cavity of the patient,
wherein measuring the level of the analyte in the sample of fluid from the mouth or other cavity of the patient determines the level of the analyte in the serum of the patient.

2. The method of claim 1, wherein the measuring the level of the analyte in the sample of fluid from the mouth or other cavity of the patient further comprises normalizing the level of the analyte to total IgG concentration in the sample of fluid from the mouth or other cavity of the patient.

3. The method of claim 1 or 2, wherein the soft tissue in the mouth of the patient comprises the gums.

4. The method of any one of claims 1-3, wherein the other cavity of the patient comprises a nostril.

5. The method of any one of claims 1-4, wherein the soft tissue in the mouth of the patient is perturbed for 0-10 seconds, 10-30 seconds, 30 seconds-1 minute, or 1-2 minutes.

6. The method of any one of claims 1-5, wherein the soft tissue in the mouth of the patient is perturbed with an oral care tool or a part thereof.

7. The method of claim 6, wherein the oral care tool comprises a manual toothbrush, an electric toothbrush, a retainer, an oral irrigator, a water flosser, a finger cot, a dental floss, a gum stimulator, a swab, a dental sponge, a swab stick, a form tip, an interdental brush, a dental scraper, a dental scaler, a dental pick, a dental stick, or a combination thereof.

8. The method of any one of claims 1-7, wherein prior to perturbing the soft tissue in a mouth of the patient, the mouth of the patient is cleaned.

9. The method of claim 8, wherein the mouth of the patient is cleaned with an oral care tool.

10. The method of any one of claims 1-9, wherein the analyte comprises an antibody or antigen-binding fragment thereof, biomarker, DNA, RNA, hormone, lipid, drug, anti-drug antibody or antigen-binding fragment thereof, other naturally occurring molecule, or a combination thereof.

11. The method of claim 10, wherein the antibody or antigen-binding fragment thereof comprises a monoclonal antibody (mAb), a monovalent antibody, an intact antibody, a bivalent antibody, a scrambled antibody, a total antibody, a polyclonal antibody, a recombinant antibody, a chimeric antibody, a humanized antibody, a camelid antibody, a CDR-grafted antibody, F(ab)2, Fv, scFv, IgGΔCH2, F(ab′)2, scFv2CH3, F(ab), VL, VH, scFv4, scFv3, scFv2, dsFv, Fv, scFv-Fc, (scFv)2, a disulfide linked Fv, a single domain antibody (dAb), a diabody, a bispecific antibody, a multispecific antibody, a dual specific antibody, an anti-idiotypic antibody, a nanobody, a VHH antibody, IgA, IgD, IgE, IgG, IgM, a modified antibody, non-depleting IgG antibodies, T-bodies, or Fc or Fab variants thereof.

12. The method of claim 10 or 11, wherein the antibody or antigen-binding fragment thereof specifically binds to a SARS-CoV-2 spike protein.

13. The method of claim 10 or 11, wherein the antibody or antigen-binding fragment thereof specifically binds to an Fc receptor.

14. The method of claim 13, wherein the Fc receptor is an IgG Fc receptor.

15. The method of claim 14, wherein the IgG Fc receptor is a neonatal Fc receptor.

16. The method of any one of claims 10-15, wherein the antibody or antigen-binding fragment thereof comprises adalimumab, infliximab, tocilizumab, vedolizumab, eculizumab, alemtuzumab, natalizumab, atezolizumab, bevacizumab, cetuximab, daratumumab, ipilimumab, nivolumab, obinutuzumab, pembrolizumab, pertuzumab, ramucirumab, rituximab, trastuzumab, golimumab, ustekinumab, denosumab, certolizumab pegol, secukinumab, alemtuzumab, or blinatumomab.

17. The method of claim 10-16, wherein the antibody or antigen-binding fragment thereof comprises natalizumab.

18. The method of claim 10, wherein the anti-drug antibody or antigen-binding fragment thereof binds to a therapeutic antibody.

19. The method of claim 18, wherein the anti-drug antibody or antigen-binding fragment thereof comprises an anti-idiotypic antibody to the therapeutic antibody.

20. The method of claim 18 or 19, wherein the anti-idiotypic antibody comprises an antigen blocking anti-idiotype antibody, a non-blocking anti-idiotype antibody, or a complex specific anti-idiotype antibody.

21. A device for determining a level of an analyte in the serum of a patient in need thereof, comprising:
a. an oral care tool or a part thereof; and
b. a lateral flow assay test.

22. The device of claim 21, wherein the oral care tool comprises a manual toothbrush, an electric toothbrush, a retainer, an oral irrigator, a water flosser, a finger cot, a dental floss, a gum stimulator, a swab, a dental sponge, a swab stick, a form tip, an interdental brush, a dental scraper, a dental scaler, a dental pick, a dental stick, or a combination thereof.

23. The device of claim 21 or 22, wherein the analyte comprises an antibody or antigen-binding fragment thereof, biomarker, DNA, RNA, hormone, lipid, drug, anti-drug antibody or antigen-binding fragment thereof, other naturally occurring molecule, or a combination thereof.

24. The device of claim 23, wherein the antibody or antigen-binding fragment thereof comprises a monoclonal antibody (mAb), a monovalent antibody, an intact antibody, a bivalent antibody, a scrambled antibody, a total antibody, a polyclonal antibody, a recombinant antibody, a chimeric antibody, a humanized antibody, a camelid antibody, a CDR-grafted antibody, F(ab)2, Fv, scFv, IgGΔCH2, F(ab′)2, scFv2CH3, F(ab), VL, VH, scFv4, scFv3, scFv2, dsFv, Fv, scFv-Fc, (scFv)2, a disulfide linked Fv, a single domain antibody (dAb), a diabody, a bispecific antibody, a multispecific antibody, a dual specific antibody, an anti-idiotypic antibody, a nanobody, a VHH antibody, IgA, IgD, IgE, IgG, IgM, a modified antibody, non-depleting IgG antibodies, T-bodies, Fc or Fab variants thereof.

25. The device of claim 23 or 24, wherein the antibody or antigen-binding fragment thereof specifically binds to a SARS-CoV-2 spike protein.

26. The device of claim 23 or 24, wherein the antibody or antigen-binding fragment thereof specifically binds to an Fc receptor.

27. The device of claim 26, wherein the Fc receptor is an IgG Fc receptor.

28. The device of claim 27, wherein the IgG Fc receptor is a neonatal Fc receptor.

29. The device of any one of claims 23-28, wherein the antibody or antigen-binding fragment thereof comprises adalimumab, infliximab, tocilizumab, vedolizumab, eculizumab, alemtuzumab, natalizumab, atezolizumab, bevacizumab, cetuximab, daratumumab, ipilimumab, nivolumab, obinutuzumab, pembrolizumab, pertuzumab, ramucirumab, rituximab, trastuzumab, golimumab, ustekinumab, denosumab, certolizumab pegol, secukinumab, alemtuzumab, or blinatumomab.

30. The device of claim 23-29, wherein the antibody or antigen-binding fragment thereof comprises natalizumab.

31. The device of claim 23, wherein the anti-drug antibody or antigen-binding fragment thereof binds to a therapeutic antibody.

32. The device of claim 31, wherein the anti-drug antibody or antigen-binding fragment thereof comprises an anti-idiotypic antibody to the therapeutic antibody.

33. The device of claim 31 or 32, wherein the anti-idiotypic antibody comprises an antigen blocking anti-idiotype antibody, a non-blocking anti-idiotype antibody, or a complex specific anti-idiotype antibody.

34. A method for treating a disease or condition in a subject in need thereof, comprising:
a. determining a level of an analyte in a serum of the patient, comprising:
i) perturbing a soft tissue in a mouth or other cavity of the patient;
ii) collecting a sample of fluid from the mouth or other cavity of the patient; and
iii) measuring the level of the analyte in the sample of fluid from the mouth or other cavity of the patient,
wherein measuring the level of the analyte in the sample of fluid from the mouth or other cavity of the patient determines the level of the analyte in the serum of the patient; and
b. administering a drug to the subject.

35. The method of claim 34, further comprising adjusting a dosage of the drug after an initial administration of the drug.

36. The method of claim 34 or 35, wherein the adjusting a dosage of the drug comprises increasing, decreasing, or maintaining the dosage of the drug.

37. The method of any one of claims 34-36, wherein the disease or condition comprises virus infection, bacterial infection, autoimmune disease, corona virus, multiple sclerosis, polyomavirus, JC virus, progressive multifocal leukoencephalopathy, Guillain-Barré syndrome, Myasthenia Gravis, flu, monkey pox, AIDS or a combination thereof.

38. The method of any one of claims 34-37, wherein the analyte comprises an antibody or antigen-binding fragment thereof, biomarker, DNA, RNA, hormone, lipid, drug, anti-drug antibody or antigen-binding fragment thereof, other naturally occurring molecule, or a combination thereof.

39. The method of claim 38, wherein the antibody or antigen-binding fragment thereof comprises a monoclonal antibody (mAb), a monovalent antibody, an intact antibody, a bivalent antibody, a scrambled antibody, a total antibody, a polyclonal antibody, a recombinant antibody, a chimeric antibody, a humanized antibody, a camelid antibody, a CDR-grafted antibody, F(ab)2, Fv, scFv, IgGΔCH2, F(ab′)2, scFv2CH3, F(ab), VL, VH, scFv4, scFv3, scFv2, dsFv, Fv, scFv-Fc, (scFv)2, a disulfide linked Fv, a single domain antibody (dAb), a diabody, a bispecific antibody, a multispecific antibody, a dual specific antibody, an anti-idiotypic antibody, a nanobody, a VHH antibody, IgA, IgD, IgE, IgG, IgM, a modified antibody, non-depleting IgG antibodies, T-bodies, Fc or Fab variants thereof.

40. The method of claims 38-39, wherein the antibody or antigen-binding fragment thereof specifically binds to a SARS-CoV-2 spike protein.

41. The method of claims 38-39, wherein the antibody or antigen-binding fragment thereof specifically binds to an Fc receptor.

42. The method of claim 41, wherein the Fc receptor is an IgG Fc receptor.

43. The method of claim 42, wherein the IgG Fc receptor is a neonatal Fc receptor.

44. The method of any one of claims 38-43, wherein the antibody or antigen-binding fragment thereof comprises adalimumab, infliximab, tocilizumab, vedolizumab, eculizumab, alemtuzumab, natalizumab, atezolizumab, bevacizumab, cetuximab, daratumumab, ipilimumab, nivolumab, obinutuzumab, pembrolizumab, pertuzumab, ramucirumab, rituximab, trastuzumab, golimumab, ustekinumab, denosumab, certolizumab pegol, secukinumab, alemtuzumab, blinatumomab, or antigen-binding fragment thereof.

45. The method of claim 38-44, wherein the antibody or antigen-binding fragment thereof comprises natalizumab.

46. A method for treating a disease or condition in a subject in need thereof, comprising:
a. determining a level of an analyte in a serum of the patient, comprising:
i) perturbing a soft tissue in a mouth or other cavity of the patient;
ii) collecting a sample of fluid from the mouth or other cavity of the patient; and
iii) measuring the level of the analyte in the sample of fluid from the mouth or other cavity of the patient,
wherein measuring the level of the analyte in the sample of fluid from the mouth or other cavity of the patient determines the level of the analyte in the serum of the patient; and
b. administering an anti-drug antibody or antigen-binding fragment thereof to the subject.

47. The method of claim 46, wherein the anti-drug antibody or antigen-binding fragment thereof binds to a therapeutic antibody.

48. The method of claim 46 or 47, wherein the anti-drug antibody or antigen-binding fragment thereof comprises an anti-idiotypic antibody to the therapeutic antibody.

49. The method of claim 47 or 48, wherein the anti-idiotypic antibody comprises an antigen blocking anti-idiotype antibody, a non-blocking anti-idiotype antibody, or a complex specific anti-idiotype antibody.
